(19) United States
(12) Patent Application Publication
(10) Pub. No.: US 2026/0174745 A1
(43) Pub. Date: Jun. 25, 2026

(54) COMPOSITIONS AND METHODS FOR TREATING NF1 AND OTHER CONDITIONS

(71) Applicants: Karen Liby, Indianapolis, IN (US); Edmund ELLSWORTH, Vicksburg, MI (US); Ana Sofia Mendes Leal, Indianapolis, IN (US)

(72) Inventors: Karen Liby, Indianapolis, IN (US); Edmund ELLSWORTH, Vicksburg, MI (US); Ana Sofia Mendes Leal, Indianapolis, IN (US)

(21) Appl. No.: 19/125,967

(22) PCT Filed: Oct. 31, 2023
(86) PCT No.: PCT/US2023/036471

§ 371 Date: Apr. 30, 2025

Related U.S. Application Data
(60) Provisional application No. 63/381,641, filed on Oct. 31, 2022.

(51) Int. Cl. A61K 31/455; A61K 31/4184; A61P 35/00

(52) U.S. Cl. CPC A61K 31/455; A61K 31/4184; A61P 35/00

(57) ABSTRACT

The disclosure relates to method for treating, among other things, NF1 or conditions resulting from NF1 function loss the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

### CROSS-REFERENCE TO RELATED APPLICATIONS

This application claims the benefit of U.S. Provisional Appl. Ser. No. 63/381,641, filed Oct. 31, 2022, which is incorporated by reference as if fully set forth herein.

### BACKGROUND

Neurofibromatosis type 1 (NF1) is an autosomal dominant genetic disease that affects approximately 1 in 3000 individuals. Patients with NF1 develop neurofibromas in many locations, including the eye and skin. While typically not medically serious, these benign lesions still have a significant deleterious impact on the lives of patients. Nearly half of patients with NF1 mutations develop plexiform neurofibromas (PNFs), and a subset of plexiform neurofibromas (˜10%) progress to highly aggressive malignant peripheral nerve sheath tumors (MPNSTs). Because no effective drugs are available to treat highly aggressive MPNSTs, poor responses to chemotherapy, and high rates of recurrence, overall survival is low for these patients.

Inactivating mutations or deletions in the NF1 tumor suppressor gene not only induce tumorigenesis but also activate inflammatory cascades that recruit immune cells to promote tumor growth. Although immune cells are rare in nerves, up to 30-50% of the cells in neurofibromas and MPNSTs are macrophages, which recruit T cells. Because these immune cells promote tumor growth, pharmacological interventions that modulate the recruitment and function of these immune cells could be valuable for inhibiting growth of neurofibromas, preventing progression of plexiform neurofibromas to MPNSTs, or treating established neurofibromas and MPNST.

The NF1 gene encodes the neurofibromin protein, which binds to and turns off the RAS protein. Loss of NF1 function results in elevated RAS signaling and activation of the downstream mitogen activated protein kinase (MAPK) cascade (RAS/RAF/MEK/ERK). NF1 loss is not only prevalent in neurofibromatosis, where it is most often inherited, but also via somatic mutations in various cancers, such as melanoma. High RAS activity in many cancers can also be attributed to mutations in genes encoding for a RAS protein. For example, activating KRAS mutations are found in 95% of pancreatic cancers and 35% of lung cancers. As with NF1 mutations, oncogenic KRAS mutations in these tumors turn on tumor-promoting inflammation and infiltration of immune cells within the tumor microenvironment (TME).

Selumetinib, a MEK inhibitor downstream of RAS activation, is the only FDA-approved treatment for patients with any NF1 manifestation. While effective in patients with plexiform neurofibromas, selumetinib is not useful for other subtypes of NF1. This drug does not alter immune cell infiltration or function and has known toxic side effects.

### SUMMARY

There is therefore a need for additional pharmacological agents for treating NF1 and conditions resulting from NF1 function loss (e.g., certain cancers, RASopathies, schwannomas, gliomas, including optical pathway gliomas, neurofibromas, intertriginous freckling, osseous lesions, and Lisch nodules). To that end, the disclosure relates to selective for retinoid X receptor (RXR) agonists and treatment modalities that include (i) the use of a single selective RXR agonist; and (ii) the combination of at least one RXR agonist and at least one inhibitor of RAS (e.g., HRAS, NRAS and KRAS), MEK, and RAF (e.g., ARAF, BRAF, and CRAF), to treat, among other conditions described herein, NF-1, conditions resulting from NF1 function loss, PNFs, and MPNSTs.

### DESCRIPTION OF THE DRAWINGS

The drawings illustrate generally, by way of example, but not by way of limitation, various embodiments discussed herein.

FIG. 1 is a scheme showing the decision funnel for identification of novel RXR agonists. In the design<act<learn process, new analogs are tested for activity, selectivity and metabolism and physical properties. The data is then used in the next design cycle.

FIG. 2 is photographs of mouse lungs and mouse lung tissue section stains collected eight weeks after initiation of Kras mutations with vinyl carbamate. A/J mice were fed control diet or RXR agonists (100 mg/kg diet or 25 mg/kg body weight) for 12 weeks. Lungs were harvested and photographed (A) and then sectioned and stained with hematoxylin and eosin (“H & E”) to evaluate tumor grade (B) as defined in collaboration with a human lung cancer pathologist. The results shown in FIG. 2 demonstrate that compound 1 reduces tumor burden and alters the histopathology when used to treat Kras-driven lung tumors. Compound 1 has the structure:

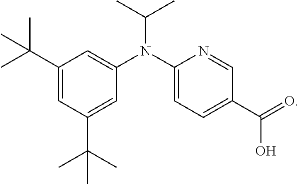 [SMILES: CC(C)N(c1cc(C(C)(C)C)cc(C(C)(C)C)c1)c1ccc(C(=O)O)cn1]

FIG. 3 is a plot of tumor burden and photographs of mouse lung tissue section stains. Eight weeks after initiation of Kras mutations, A/J mice were fed control diet or compound 1 (25 mg/kg) for 12 weeks. Lungs were sectioned and stained with H & E or by immunohistochemistry (“IHC”) to evaluate tumor burden or biomarkers, respectively. The results in FIG. 3 demonstrate that compound 1 reduces tumor burden and p-ERK and CD206 expression when used to treat Kras-driven lung tumors.

FIG. 4 is plots showing data collected from lung tumors induced in A/J mice as described in FIG. 3. After 12 weeks of treatment with compound 1 (25 mg/kg) or control diet, lungs were harvested. Immune cell populations in the lungs were analyzed by flow cytometry using an optimized multi-colored panel of antibodies. The results in FIG. 4 demonstrate that compound 1 alters T cell populations in lung tumors.

FIG. 5 is plots and stains obtained from MMTV-neu mice with established mammary tumors, which were treated with compound 1 for 10 days. Tumor size (A) was evaluated by calipers, immune cell populations by flow cytometry (B) and IHC (C). n=8 mice/group in A-C and n=4/group for quantification (D) of PCNA and p-ERK optical density. The data in FIG. 5 compound 1 reduces tumor burden and p-ERK and alters T cell populations in mammary tumors.

FIGS. 6A and 6B are a chromatogram and a plot, respectively, generated from experiments on NF1-ipNF 95.6 neurofibroma cells treated with 50 nM selumetinib (Sel), 5 nM trametinib (Tra, an alternative MEK inhibitor used as a control), 200 nM compound 1, or the combination for 3 hours. Protein expression was evaluated by western blotting and LiCOR imaging (A). In B, cells were treated with 200 nM MSU42011, 100 nM Sel or the combination for 72 hrs, and cell viability measured using an MTT assay. *, P<0.05 vs. other groups. The results in FIGS. 6A and 6B demonstrate that compound 1, alone and in combination with selumetinib, decreases the expression of p-ERK and reduces viability of NF1-ipNF 95.6 neurofibroma cells.

FIG. 7 is a plot of CCL2 expression and a cartoon of the differentiation of THP1 monocytes into macrophages with 50 ng/ml PMA for 3 days, followed by treatment with conditioned medium (CM) from ipNF95.6 neurofibroma cells for an additional 24 hrs. Left panel (-): undifferentiated THP-1 monocytes. Right panel: PMA-differentiated THP-1 macrophages. CCL2 mRNA expression was evaluated by qPCR. Data represent means±standard deviations of three independent experiments. The results in FIG. 7 demonstrate that conditioned media (CM) from human neurofibroma cells increases expression of CCL2 mRNA in THP1 macrophages.

FIG. 8 is a plot showing results of experiments involving THP1 human monocytes that were differentiated into macrophages with 50 ng/ml PMA for 3 days, followed by treatment with conditioned medium (CM) from ipNF95.6 neurofibroma cells and drugs (50 nM selumetinib (Sel), 200 nM compound 1, or the combination) for an additional 24 hrs. CCL2 expression was evaluated by qPCR and normalized to the DMSO control without treatment with PMA+CM (not shown). Data represent means±standard deviations of two independent experiments, p<0.05 vs. DMSO control. The results in FIG. 8 demonstrate that the combination of compound 1 and selumetinib decreases CCL2 mRNA expression in human macrophages stimulated with conditioned media from human neurofibroma cells.

FIG. 9 is a plot showing the results from additional studies conducted where 1×10⁶ LL2 cells were implanted subcutaneously into the flank of 7-8 week old male C57/B16 mice. This experiment compares single agent RXR (compound 1) with single agent MEK inhibitor (selumetinib) and the combination in a RAS-activated model. MEK inhibitors are the standard of care for NF1-related conditions and a class also approved by various cancers, including melanoma. Activating genetic alternations in RAS pathway include, but are not limited to, mutations in KRAS, NRAS, HRAS, BRAF, CRAF, as well as loss of function deletions or mutations in NF1, as is the case in NF1 patients.

FIG. 10A is a plot showing the results where THP1 monocytes were differentiated into macrophages with 50 ng/ml PMA for 3 days, followed by treatment with CM from human PNF cells (ipNF95.6) and drugs (50 nM selumetinib, 200 nM compound 1, or the combination) for an additional 24 hrs.

FIG. 10B is a plot showing the results where BMDM were differentiated into macrophages with 20 ng/ml M-CSF for 5 days, followed by treatment with CM from mouse PNF cells (NF1f/f Cre+) and drugs (50 nM selumetinib, 200 nM compound 1, or the combination) for an additional 24 hrs. CCL2 mRNA expression was evaluated by qPCR and normalized to the DMSO control without CM treatment (not shown). Data represent means±standard deviations. * p<0.05, ** p<0.01 compared with vehicle treatment confirm the translation of compound 1 and MEK inhibitor data from an in vivo RAS-mutant lung model into an NF1 disease relevant in vitro model.

FIGS. 11A and 11B are plots showing the results of dose ranging studies. Two-way ANOVA w/Tukey HSD results are shown in FIG. 11A. The data in FIG. 11B show triglyceride levels measured under the same conditions as for the data in FIG. 11A. FDA-approved bexarotene is not active against the tumors but still raises triglycerides.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Neurofibromatosis type 1 (NF1) is a common genetic disease that predisposes approximately 50% of affected individuals to develop plexiform neurofibromas (PNFs). PNF is the most common benign Schwann cell tumor in patients with NF1, which can progress to MPNST, a highly aggressive tumor and a major cause of death in patients. In patients with a NF1 mutation, hyperactive Ras signaling promotes cell proliferation through downstream activation of MAPK and PI3K/AKT pathways. Moreover, macrophages make up approximately 30-50% of the cells in both PNF and MPNST.

PNF can progress to highly aggressive malignant peripheral nerve sheath tumors (MPNSTs) in approximately 10% of patients. NF1 is caused by mutations in the tumor suppressor gene NF1, which encodes for neurofibromin, a negative regulator of RAS activity. Selumetinib, a specific inhibitor of MEK1/2, is the only FDA-approved drug for NF1-associated PNFs. However, as discussed previously herein, selumetinib is not active against MPNSTs and have dose-limiting side effects.

Deficiency of the NF1 gene not only promotes tumorigenesis but also has broad effects on the immune cells and cytokine signaling driven by hyperactive RAS signaling. Because macrophages account for almost half of cells in NF1 lesions and their infiltration highly correlates with disease progression, it is believed that targeting tumor-promoting immune cells could be an alternative approach for NF1 treatment.

Described herein are RXR agonists that can, among other things, reduce tumor growth (for example, Kras-driven cancers) by decreasing p-ERK expression, reducing tumor-promoting immune cells like CD206+ macrophages and regulatory T cells, and increasing activated cytotoxic T cells. Compositions and methods of using the RXR agonists are also described herein. Such compositions and methods are useful for treating, among other conditions, NF-1, conditions resulting from NF1 function loss, plexiform neurofibromas (PNFs), and malignant peripheral nerve sheath tumors (MPNSTs).

For example, the retinoid X receptor (RXR) agonists used in the methods described herein, can include compounds of the Formula (I), which are disclosed in Published PCT Appl. No. WO2020/150668, which is incorporated by reference as if fully set forth herein:

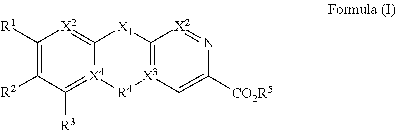

or a pharmaceutically acceptable salt thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl or heterocyclyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹ is alkyl;
R² is H, alkyl (e.g., cycloalkyl, such as cyclopropyl) or alkoxy, provided that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group. The compounds of the Formula (I) can be at least disubstituted at R¹, R², R³, and R⁴.

The compound of Formula (I) can have R² and R³, together with the carbon atoms to which they are attached, form a cycloalkyl group. Or the compound of Formula (I) can have R³ and R⁴, together with the carbon atoms to which they are attached, form a cycloalkyl group.

The X¹ group can include C═C(R⁶R⁷) or NR⁸, as indicated above, where R⁶, R⁷, and R⁸ are each independently H or alkyl. However, in some cases the X¹ group can be NR⁸. But in some cases, the X¹ group can be C═C(R⁶R⁷).

The R¹ group can be alkyl, and R³ can be H or alkyl, as indicated above. However, in some cases both of R¹ and R³ can each be alkyl. But in some cases, R¹ can be alkyl and R² can be H or alkoxy.

The R² group can be H, alkyl or alkoxy, as indicated above. However, in some cases, the R² group can be H (hydrogen).

The X² group can be nitrogen (N) or CR⁹, as indicated above, wherein R⁹ is H or R⁸ and R⁹, together with the atoms to which they are each attached, form a heterocyclyl group. Also, as indicated above, X³ can be CH or N. However, in some cases X² can be N and X³ can be CH. But in some cases, X² can be CH and X³ can be CH.

The R⁴ group can be H, alkyl or alkoxy, as indicated above. However, in some cases, the R⁴ group can be hydrogen (H). But in some cases, R¹ can be alkyl and R⁴ can be alkoxy.

The R⁵ group can be H or alkyl, as indicated above. However, in some cases, the R⁵ group can be hydrogen (H).

Compounds contemplated for use in the methods described herein can also include compounds of the Formula (II)-(XVI):

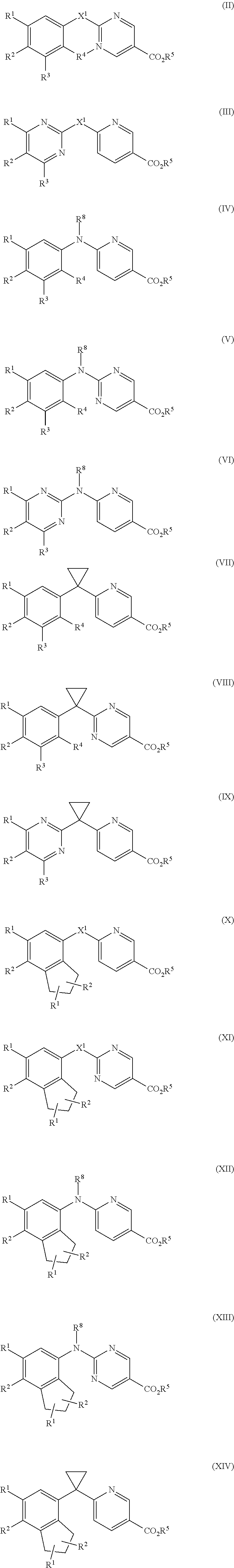

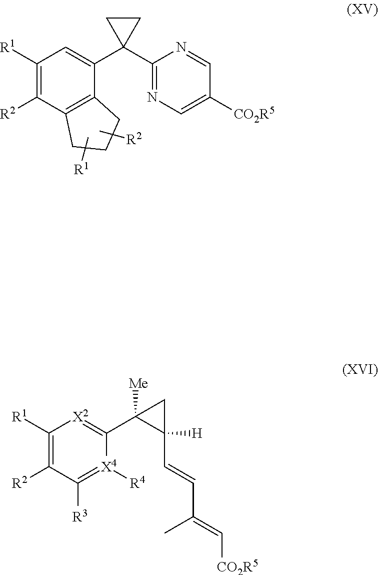

or a pharmaceutically acceptable salt thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹ is alkyl;
R² is H, alkyl or alkoxy, provided in some cases that when X² is N and X³ is CH, then
R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group. The compounds of any of the foregoing formulae can be at least disubstituted at R¹, R², R³, and R⁴.

Examples of compounds contemplated for use in the methods described herein can also include the following, or a pharmaceutically acceptable salt thereof:

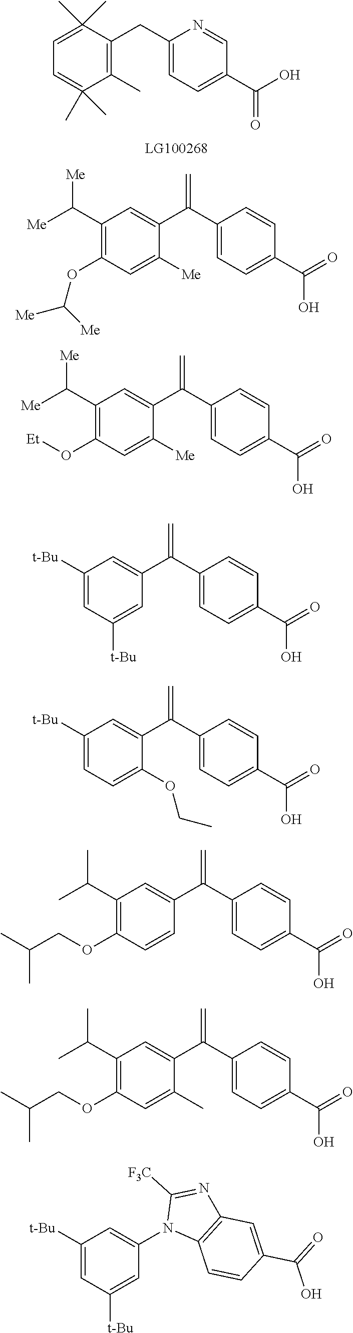

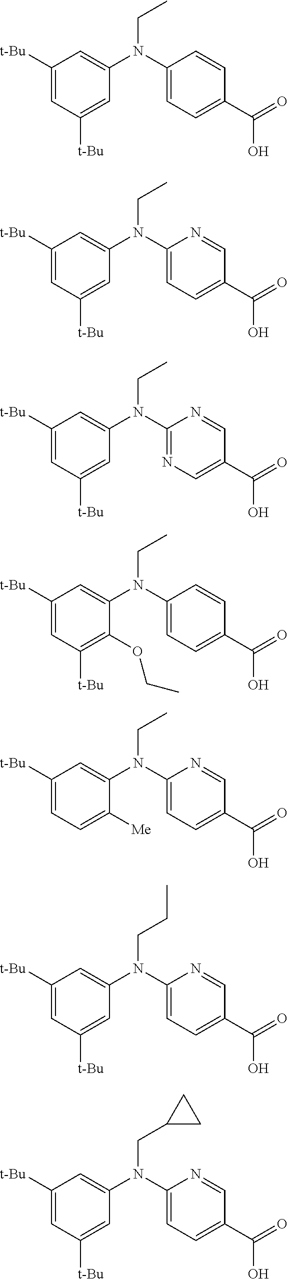

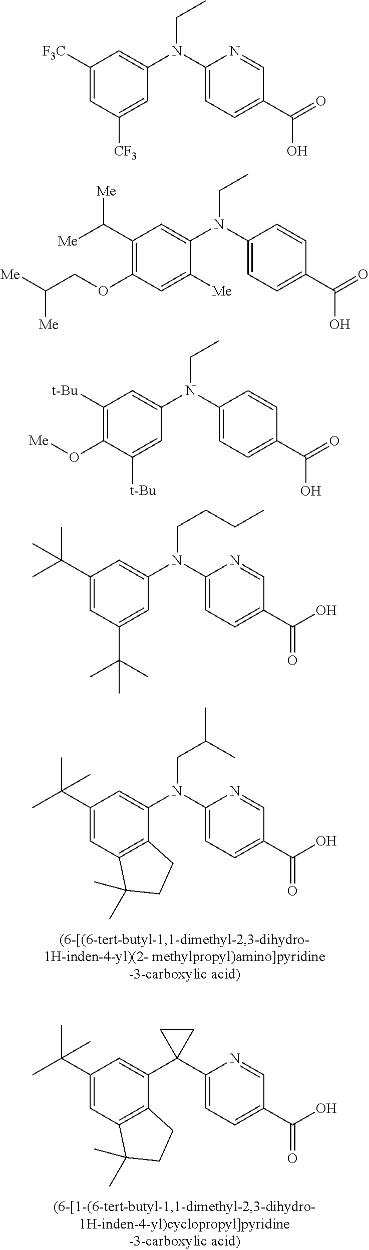

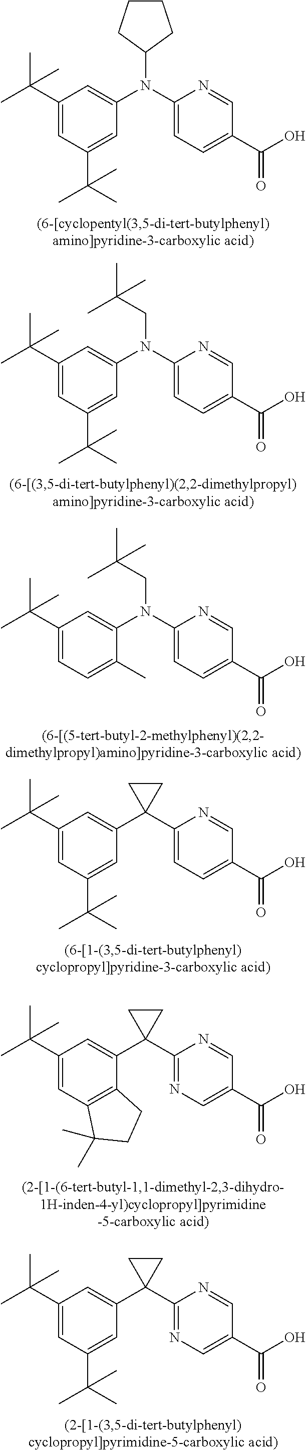 [SMILES: CC(C)(C)c1cc(N(c2ccc(C(=O)O)cn2)C2CCCC2)cc(C(C)(C)C)c1 ; CC(C)(C)CN(c1cc(C(C)(C)C)cc(C(C)(C)C)c1)c1ccc(C(=O)O)cn1 ; Cc1ccc(C(C)(C)C)cc1N(CC(C)(C)C)c1ccc(C(=O)O)cn1 ; CC(C)(C)c1cc(C(C)(C)C)cc(C2(c3ccc(C(=O)O)cn3)CC2)c1 ; CC(C)(C)c1cc2c(c(C3(c4ncc(C(=O)O)cn4)CC3)c1)CCC2(C)C ; CC(C)(C)c1cc(C(C)(C)C)cc(C2(c3ncc(C(=O)O)cn3)CC2)c1]

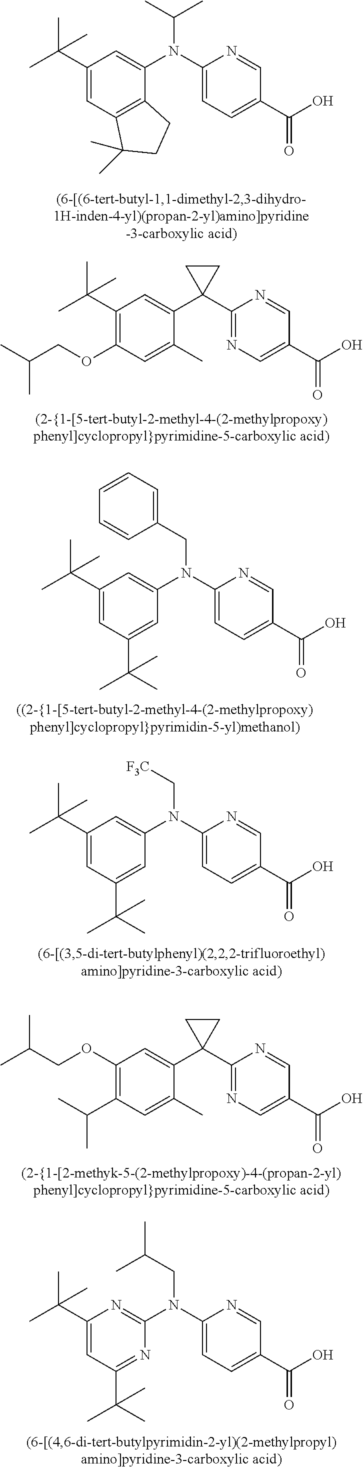 [SMILES: CC(C)N(c1ccc(C(=O)O)cn1)c1cc(C(C)(C)C)cc2c1CCC2(C)C ; Cc1cc(OCC(C)C)c(C(C)(C)C)cc1C1(c2ncc(C(=O)O)cn2)CC1 ; CC(C)(C)c1cc(N(Cc2ccccc2)c2ccc(C(=O)O)cn2)cc(C(C)(C)C)c1 ; CC(C)(C)c1cc(N(CC(F)(F)F)c2ccc(C(=O)O)cn2)cc(C(C)(C)C)c1 ; Cc1cc(C(C)C)c(OCC(C)C)cc1C1(c2ncc(C(=O)O)cn2)CC1 ; CC(C)CN(c1ccc(C(=O)O)cn1)c1nc(C(C)(C)C)cc(C(C)(C)C)n1]

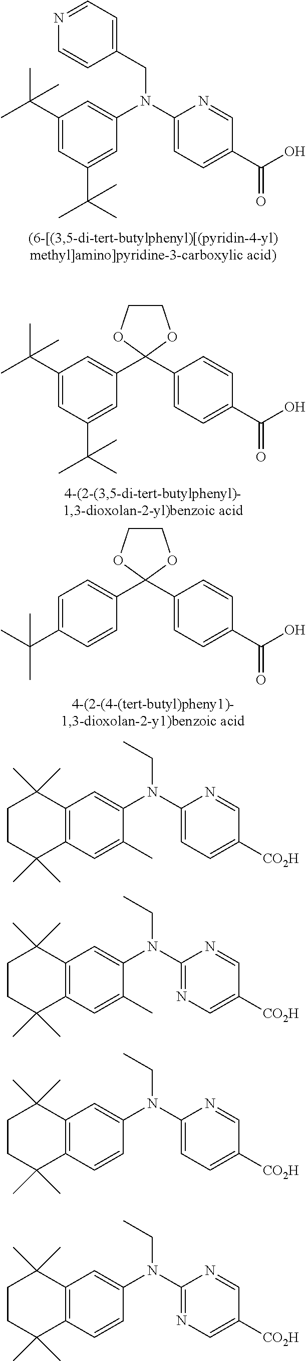

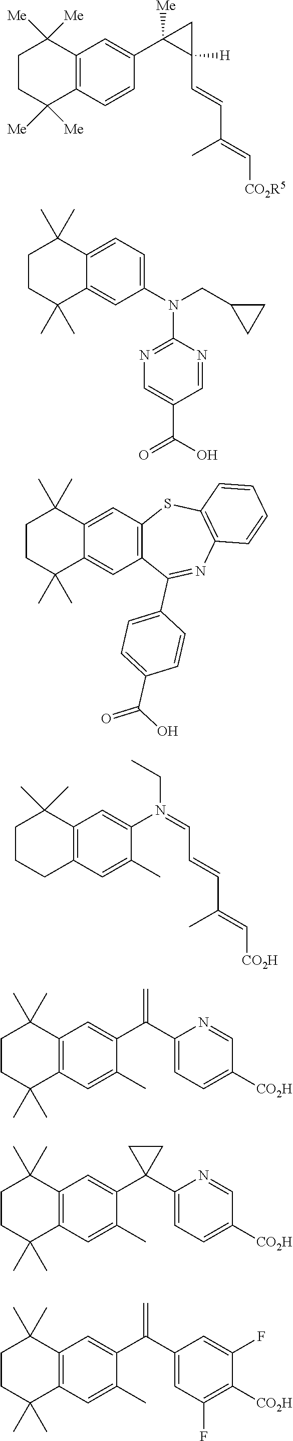

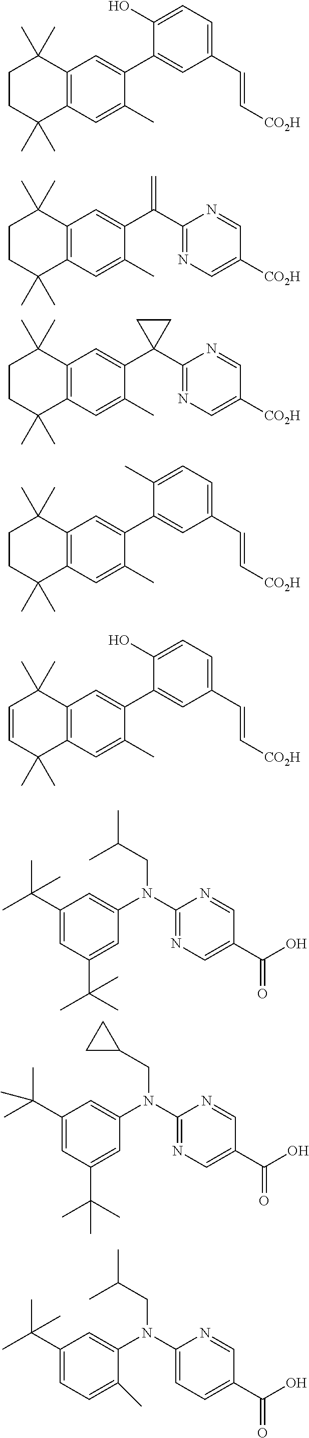

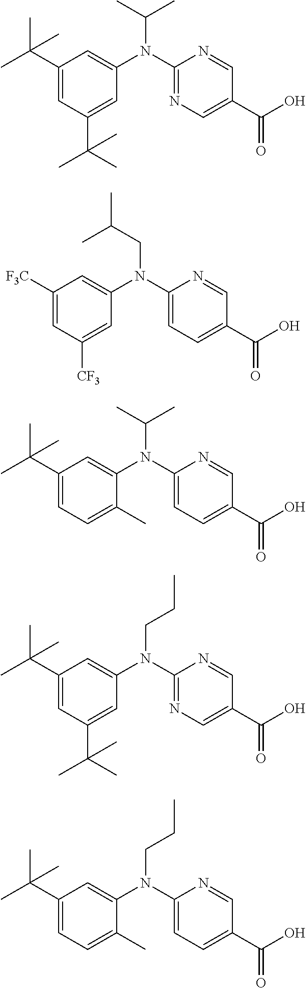

or a combination thereof.

Compounds contemplated herein for the various methods and uses described herein also include compounds of the Formula (I):

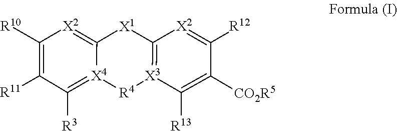

or a pharmaceutically acceptable salt thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl, cycloalkenyl or heterocyclyl group;
each X² is, independently, N or CR⁹, wherein R⁹ is H or R⁸ and R⁹, together with the atoms to which they are each attached, form a heterocyclyl group;
X³ is CH or N;
X⁴ is N or C;
R¹⁰ and R¹¹ form a ring, such as cycloalkyl, cycloalkenyl or a heterocyclyl ring;
R⁴ is absent, H, alkyl or alkoxy;
R³ is H or alkyl;
R⁵ is H or alkyl;
or R² and R³ or R³ and R⁴, together with the carbon atoms to which they are attached, form a cycloalkyl group; and
R¹² and R¹³ are each, independently, H or halo, such as chloro, bromo or fluoro . . .

Compounds contemplated herein for the various methods and uses described herein also include compounds of the formulae:

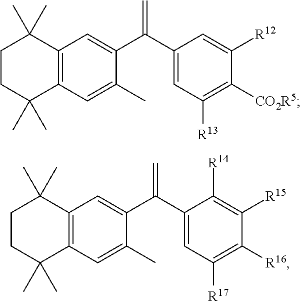

wherein R¹⁴, R¹⁵, R¹⁶, and R¹⁷ are each, independently, H, CO₂R⁵, NO₂ or halo (e.g., F);

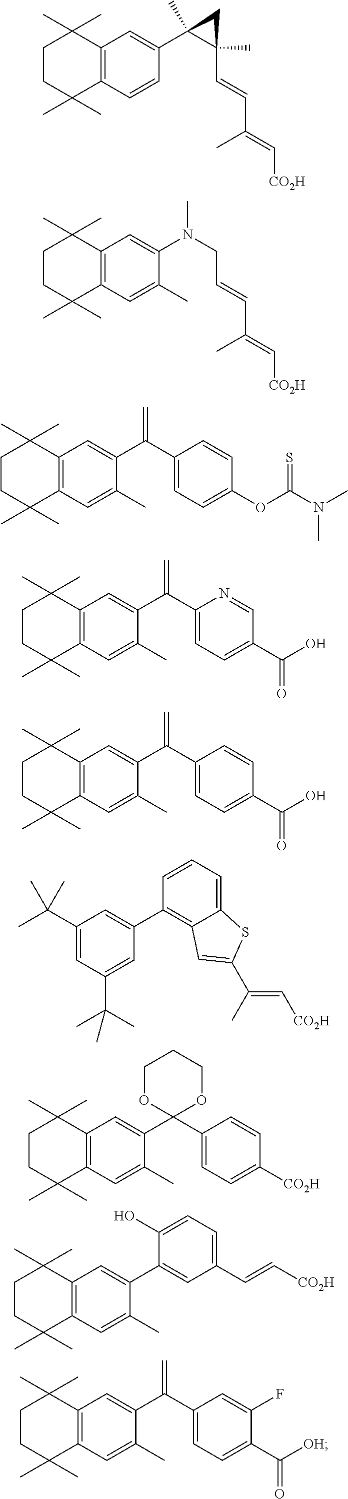

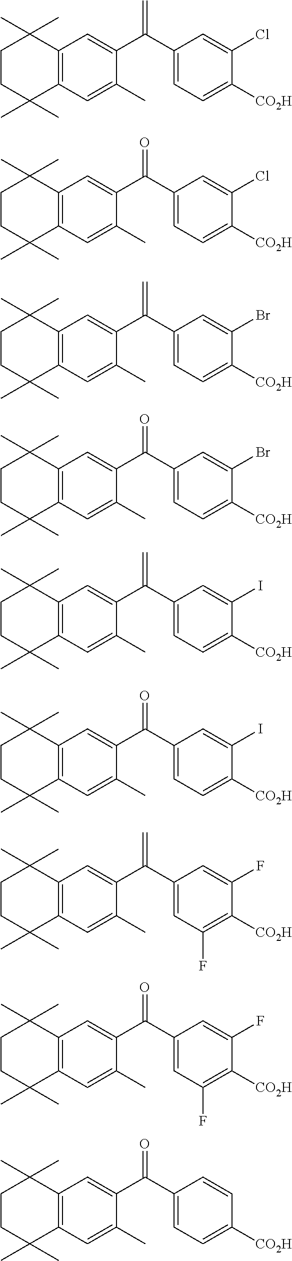

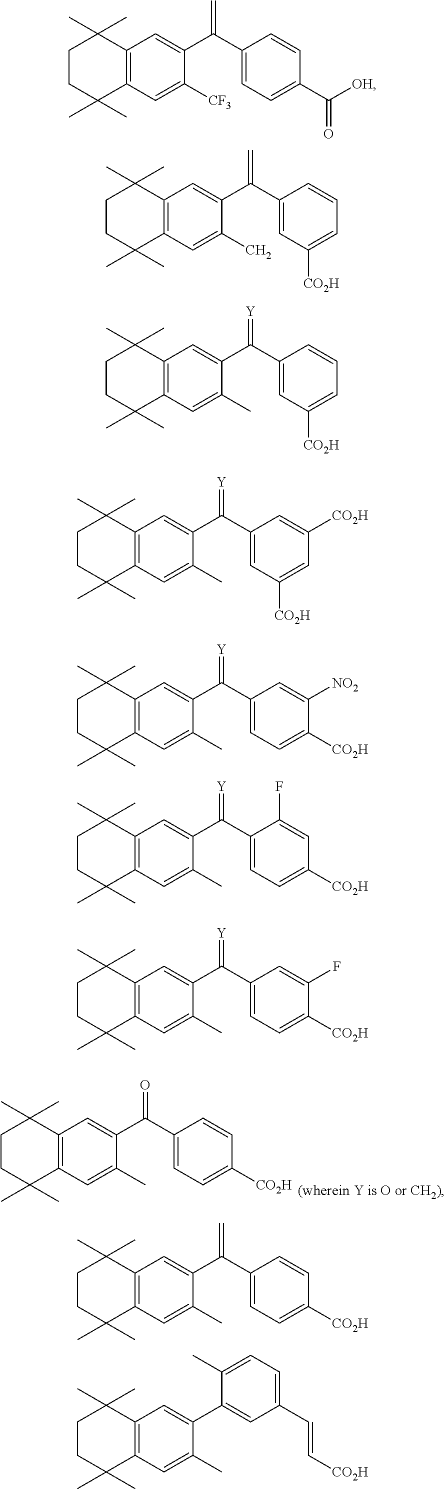

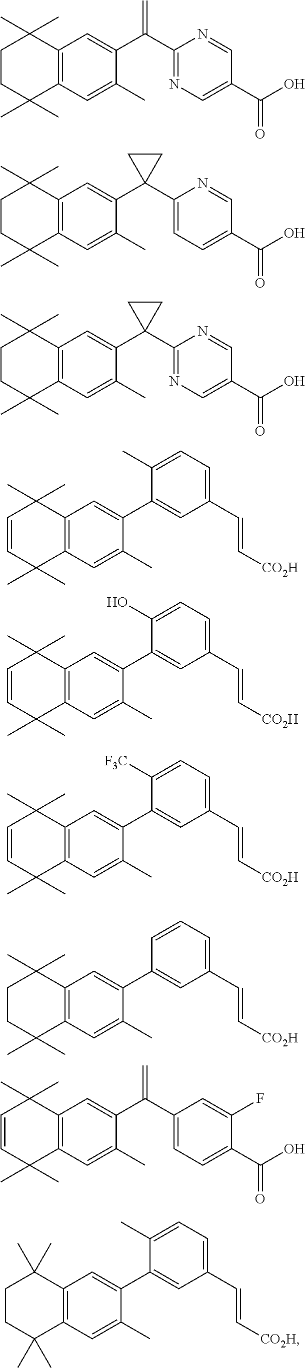

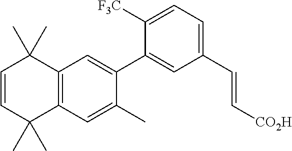 [SMILES: Cc1cc2c(cc1-c1cc(/C=C/C(=O)O)ccc1C(F)(F)F)C(C)(C)C=CC2(C)C]

and pharmaceutically acceptable salts thereof.

Examples of compounds contemplated for use in the methods described herein can also include compounds of the Formula (I):

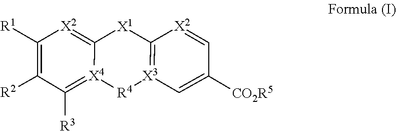

or a pharmaceutically acceptable salt, wherein:
X¹ is NR⁸, wherein R⁸ is H or alky;
each X² is, independently, N or CR⁹, wherein R⁹ is H or alkyl;
X³ is CH or N;
X⁴ is N or C;
R¹ is alkyl;
R² is H, alkyl (e.g., cycloalkyl, such as cyclopropyl) or alkoxy, provided that when X² is N and X³ is CH, then R² is not isobutoxy;
R³ is H or alkyl;
R⁴ is absent, H, alkyl or alkoxy; and
R⁵ is H or alkyl;
or R² and R³ or R³ and R⁴, together with the carbon atoms to which they are attached, form a cycloalkyl group;
wherein the compound of Formula (I) is at least disubstituted with R¹-R⁴.

The compound of Formula (I) can have R² and R³, together with the carbon atoms to which they are attached, form a cycloalkyl group. Or the compound of Formula (I) can have R³ and R⁴, together with the carbon atoms to which they are attached, form a cycloalkyl group.

The R¹ group can be alkyl, and R³ can be H or alkyl, as indicated above. However, in some cases both of R¹ and R³ can each, independently be, alkyl. But in some cases, R¹ can be alkyl and R² can be H or alkoxy. R¹ and R³ can, for example, each independently be C₁-C₆-alkyl groups, such as t-butyl groups.

The R² group can be H, alkyl or alkoxy, as indicated above. However, in some cases, the R² group can be H.

The X² group can be N or CR⁹, as indicated above, wherein R⁹ is H or R⁸ and R⁹, together with the atoms to which they are each attached, form a heterocyclyl group. Also, as indicated above, X³ can be CH or N. However, in some cases X² can be N and X³ can be CH. But in some cases, X² can be CH and X³ can be CH.

The R⁴ group can be H, alkyl or alkoxy, as indicated above. However, in some cases, the R⁴ group can be H. But in some cases, R¹ can be alkyl and R⁴ can be alkoxy.

The R⁵ group can be H or alkyl, as indicated above. However, in some cases, the R⁵ group can be H.

RXR agonists for use in the methods described herein, also include compounds of the Formula (II)-(VII):

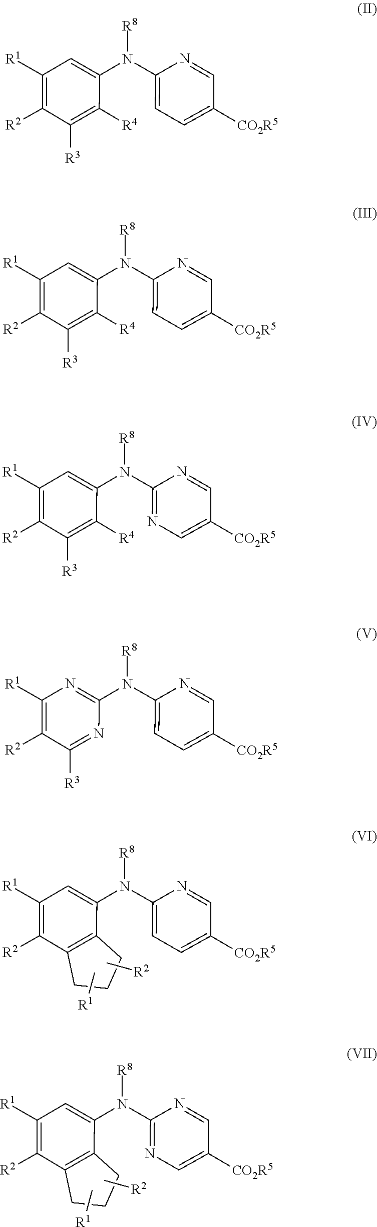

or a pharmaceutically acceptable salt, polymorph, prodrug, solvate or clathrate thereof, wherein:
R¹ is alkyl;
R² is H, alkyl (e.g., cycloalkyl, such as cyclopropyl) or alkoxy, provided that when X² is N and X³ is CH, then R² is not isobutoxy;
R³ is H or alkyl;
R⁴ is absent, H, alkyl or alkoxy; and
R⁵ is H or alkyl;
or R² and R³ or R³ and R⁴, together with the carbon atoms to which they are attached, form a cycloalkyl group; and
R⁸ is alkyl;
wherein each compound is at least disubstituted with R¹-R⁴.

An example of compounds that can be useful RXR agonists includes compound 1:

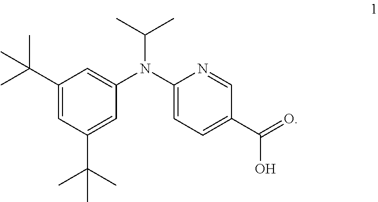 [SMILES: CC(C)N(c1cc(C(C)(C)C)cc(C(C)(C)C)c1)c1ccc(C(=O)O)cn1]

or a pharmaceutically acceptable salt, polymorph, prodrug, solvate or clathrate thereof.

The disclosure also contemplates (i) the use of a single selective RXR agonist (whether of the Formulae (I)-(XVI) or not); and (ii) the combination of at least one RXR agonist (whether of the Formulae (I)-(XVI) or not) and at least one inhibitor of RAS, MEK or BRAF to treat, among other conditions, NF-1, conditions resulting from NF1 function loss, plexiform neurofibromas (PNFs), malignant peripheral nerve sheath tumors (MPNSTs), certain cancers, RASopathies (e.g., neurofibromatosis type 1, Noonan syndrome, Noonan syndrome with multiple lentigines, capillary malformation-arteriovenous malformation syndrome, Costello syndrome, cardio-facio-cutaneous syndrome, and Legius syndrome and combinations thereof), schwannomas, gliomas (including optical pathway gliomas, low-grade glioma, and high-grade glioma), neurofibromas, intertriginous freckling, osseous lesions, and Lisch nodules. Examples of cancers that can be treated with the compounds of the Formulae (I)-(XVI) include rhabdomyosarcoma, neuroblastoma, juvenile myelomonocytic leukemia, gastronintestinal stromal tumors, carcinoid, cancer, breast endocrine cancers (including pheochromocytoma and neuroendocrine tumors), melanoma, acute lymphoblastic leukemia, ovarian cancer, prostate cancer, meningioma, and undifferentiated pleomorphic sarcoma.

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. An example of a substituted alkyl also includes halo alkyl, such as trifluoromethyl. An example of substituted alkyl also includes cycloalkyl substituted alkyl, such as cyclopropyl methyl. An example of a substituted alkyl is arylalkyl, such as benzyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃) CH₂— and —CH₂CH(CH₃) CH₂—. Examples of cyclic alkyl groups include cyclopropyl, cyclobutyl, cyclopentyl, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted with one or more groups such as amino, hydroxy, cyano, carboxy, nitro, thio, alkoxy, or halogen groups. Thus, in some embodiments, alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl. The term “alkyl” also generally refers to alkyl groups that can comprise one or more heteroatoms in the carbon chain. Thus, for example, “alkyl” also encompasses groups such as —[(CH₂)ₚO]_{q}H and the like.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)-alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from one or more groups such as monoalkylamino, dialkylamino, cyano, acetyl, amido, carboxy, nitro, alkylthio, alkoxy, or halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), CH₂C≡CH, CH₂C≡C(CH₃), and CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀) aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀) aryl encompasses mono- and polycyclic (C₆-C₂₀) aryl groups, including fused and non-fused polycyclic (C₆-C₂₀) aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise, a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

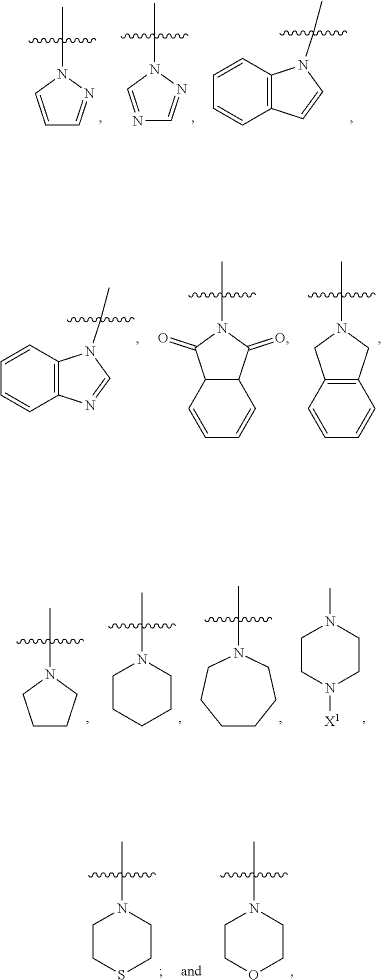

wherein X⁵ represents H, (C₁-C₂₀)-alkyl, (C₆-C₂₀) aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyloxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amine” and “amino” as used herein refers to a substituent of the form-NH₂, —NHR, —NR₂, —NR₃+, wherein each R is independently selected, and protonated forms of each, except for—NR³+, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to a nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “substituted” as used herein refers to a group that is substituted with one or more groups including, but not limited to, the following groups: halogen (e.g., F, Cl, Br, and I), R, OR, OC(O)N(R)₂, CN, NO, NO₂, ONO₂, azido, CF₃, OCF₃, methylenedioxy, ethylenedioxy, (C₃-C₂₀) heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH) N(R)₂, C(O)N(OR)R, or C(═NOR) R wherein R can be hydrogen, (C₁-C₂₀)alkyl or (C₆-C₂₀) aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. In yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

One aspect of the invention is a method that includes administering to a subject (e.g., an animal or human) a compound or composition described herein. The subject so treated can be in need of such administration. The administration can also be to inhibit the onset of disease. The subject can be any type of animal, for example, a human, a domesticated animal, an animal involved in experimental research, or a zoo animal. Examples of animals that can be administered the compositions and compounds described herein can include mice, rats, dogs, cats, rabbits, goats, sheep, cattle, horses, swine, and the like. In some cases, the compositions and compounds described herein can be administered to a human subject or a laboratory animal.

The compositions and methods are useful for treating diseases and conditions, as well as for inhibiting the onset of diseases and conditions (prophylactic).

The disclosure also relates to compositions containing one or more of the compounds described herein. The compositions of the invention can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

Administration of one or more compounds in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the compounds and compositions of the invention may be essentially continuous over a preselected period of time or may be in a series of spaced doses. Both local and systemic administration is contemplated. The disclosure also contemplates (i) the use of a single selective RXR agonist (whether of the Formulae (I)-(XVI) or not); or (ii) the combination of one or more RXR agonists (whether of the Formulae (I)-(XVI) or not) and at least one inhibitor of RAS, MEK, and BRAF in the methods described herein. For (ii) each RXR agonist (whether of the Formulae (I)-(XVI) or not) and/or each inhibitor of RAS, MEK, and BRAF can be formulated together or separately, administered together or separately. Thus, for example, an inhibitor of RXR can be formulated together with at least one inhibitor of RAS; an inhibitor of RXR can be formulated together with at least one inhibitor of MEK; an inhibitor of RXR can be formulated together with at least one inhibitor of BRAF; or an inhibitor of RXR can be formulated together with at least one inhibitor of RAS and at least one inhibitor of MEK and at least one inhibitor of BRAF.

Examples of RAS inhibitors include AMG-510, MRTX 849, captopril, imidapril, zofenopril, candesartan, delapril, telmisartan, aliskiren, moexipril, enalapril, valsartan, fosinopril, irbesartan, perindopril, quinapril, ramipril, eprosartan, olmesartan, trandolapril, losartan, azilsartan, lisinopril, spirapril, benazepril, cilazapril and those disclosed in WO2018/068017, WO2018/140513, WO2018/140514, and WO2020/173938, each of which is incorporated by reference as if fully set forth herein, and pharmaceutically acceptable salts of each of the foregoing.

Examples of MEK inhibitors include refametinib, selumetinib, trametinib, cobimetinib, pimasertib, RO-4987655, RO5126766, WX-554, HL-085, PD-0325901, MEK 162, AZD-8330, TAK-733, and GDC-0623 or pharmaceutically acceptable salts of each of the foregoing. See Molecules. 2017 October; 22 (10): 1551, which is incorporated by reference as if fully set forth herein.

Examples of BRAF inhibitors include vemurafenib, dabrafenib, encorafenib, and binimetinib or pharmaceutically acceptable salts of each of the foregoing.

To prepare the composition, compounds and other agents are synthesized or otherwise obtained, purified as necessary or desired. Pharmaceutically acceptable salts of the compounds can be prepared. The compounds, salts thereof, and other agents can be suspended in a pharmaceutically acceptable carrier and/or lyophilized or otherwise stabilized. The compounds and combinations thereof can be adjusted to an appropriate concentration, and optionally combined with other agents. The absolute weight of a given compound and/or other agents included in a unit dose can vary widely.

As used herein, the term “therapeutically effective amount” means (unless specifically stated otherwise) a quantity of a compound (e.g., of the Formula (I)) which, when administered either one time or over the course of a treatment cycle affects the health, well-being or mortality of a subject (e.g., and without limitation, delays the onset of and/or reduces the severity of one or more of the symptoms or signs associated with the conditions described herein). Useful dosages of the compounds described herein can be determined, for example, by comparing their in vitro activity and in vivo activity in animal models. Methods of the extrapolation of effective dosages in mice and other animals to human subjects are known in the art. Indeed, the dosage of the compound can vary significantly depending on the condition of the host subject, how advanced the pathology is, the route of administration of the compound and tissue distribution, and the possibility of co-usage of other prophylactic or therapeutic treatments (such as radiation therapy or additional drugs in combination therapies). The amount of the composition required for use in treatment (e.g., the therapeutically amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise. An effective (e.g., prophylactically or therapeutically) amount or dose can range, for example, from about 0.05 mg/kg of patient body weight to about 250.0 mg/kg of patient body weight, 10 mg/kg of patient body weight to about 250.0 mg/kg, 10 mg/kg of patient body weight to about 100 mg/kg, about 0.01 mg/kg of patient body weight to about 5.0 mg/kg of patient body weight, 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. The total effective (e.g., prophylactically or therapeutically) amount of the compounds described herein can be administered in a single dose or divided doses and can, at the practitioner's discretion, fall outside of the typical range given herein.

The compounds of the disclosure can be administered in an effective (e.g., prophylactically or therapeutically) amount of from about 0.5 g/m to about 500 mg/m², from about 0.5 g/m² to about 300 mg/m², or from about 100 g/m² to about 200 mg/m². In other embodiments, the amount can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². The total amount can be administered in a single dose or divided doses and can, at the physician's discretion, fall outside of the typical range given herein. These amounts are based on m² of body surface area.

It will be appreciated that the amounts of compounds and/or other agents for use in treatment will vary not only with the particular carrier selected but also with the route of administration, the nature of the cancer condition being treated and the age and condition of the patient. Ultimately the attendant health care provider can determine proper dosage. In addition, a pharmaceutical composition can be formulated as a single unit dosage form.

Thus, one or more suitable unit dosage forms comprising the compound(s) and/or agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The compound(s) and/or agents may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the compound(s) with liquid carriers, solid matrices, semi-solid carriers, finely divided solid carriers or combinations thereof, and then, if necessary, introducing or shaping the product into the desired delivery system. For example, the compound(s) can be linked to a convenient carrier such as a nanoparticle, albumin, polyalkylene glycol, or be supplied in prodrug form. The small compound(s) and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of compound(s) can also involve parenteral or local administration of the in an aqueous solution or sustained release vehicle.

Thus, while the compound(s) and/or other agents can sometimes be administered in an oral dosage form, that oral dosage form can be formulated to protect the compound(s) from degradation or breakdown before the compound(s) and combinations thereof provide therapeutic utility. For example, in some cases the compound(s) and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The compound(s) can be formulated in dry form (e.g., in freeze-dried form), in the presence or absence of a carrier. If a carrier is desired, the carrier can be included in the pharmaceutical formulation, or can be separately packaged in a separate container, for addition to the compound(s) that are packaged in dry form, in suspension or in soluble concentrated form in a convenient liquid.

Compound(s) can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as other RXR agonists, chemotherapeutic agents, anti-viral agents, antibacterial agents, antimicrobial agents and/or preservatives. Examples of additional therapeutic agents that may be used include, but are not limited to: MEK inhibitors, such as selumetinib; RXR agonists, such as those disclosed in WO2020/150668, which is incorporated by reference as if fully set forth herein; anti-PD-L1 antibodies, alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), nab-paclitaxel, docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The inhibitors can also be used in conjunction with radiation therapy.

The compositions can be administered daily, twice-daily (BID), thrice-daily (TID), or some other administration regimen. In some cases, the compositions can be administered three times a week, twice a week, or once a week.

The compositions and methods described herein can provide in vivo activity better than currently available rexinoids such as bexarotene for the treatment of Kras mutated cancers. The compositions and methods described herein can increase >10% increase in PD-L1 protein by more than 10% in vitro and by more than 15% in vivo. In contrast, bexarotene does not increase expression of this checkpoint inhibitor. In addition, the compositions and methods described herein provide no or only minimal elevation of triglycerides relative to background indicating that the rexinoids described herein have an acceptable safety profile for development.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

The disclosure also relates to the following enumerated Embodiments:

Embodiment 1 relates to a method for treating NF1 or conditions resulting from NF1 function loss the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

Embodiment 2 relates to the method of Embodiment 1, wherein the RXR agonist can be formulated together or separately with at least one inhibitor of RAS, MEK, and BRAF or administered together or separately with at least one inhibitor of RAS, MEK, and BRAF.

Embodiment 3 relates to the method of Embodiment 1 or 2, wherein the RXR agonist is a compound of the Formula (I):

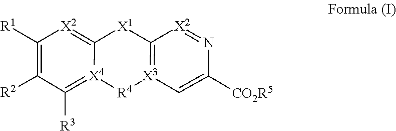

or a pharmaceutically acceptable salt, polymorph, prodrug, solvate or clathrate thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl or heterocyclyl group;
each X² is, independently, N or CR⁹, wherein R⁹ is H or alkyl or R⁸ and R⁹, together with the atoms to which they are each attached, form a heterocyclyl group;
X³ is CH or N;
X⁴ is N or C;
R¹ is alkyl;
R² is H, alkyl or alkoxy, provided in some cases that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group.

Embodiment 4 relates to the compound of Embodiments 1-3, wherein the RXR agonist is a compound of the formula:

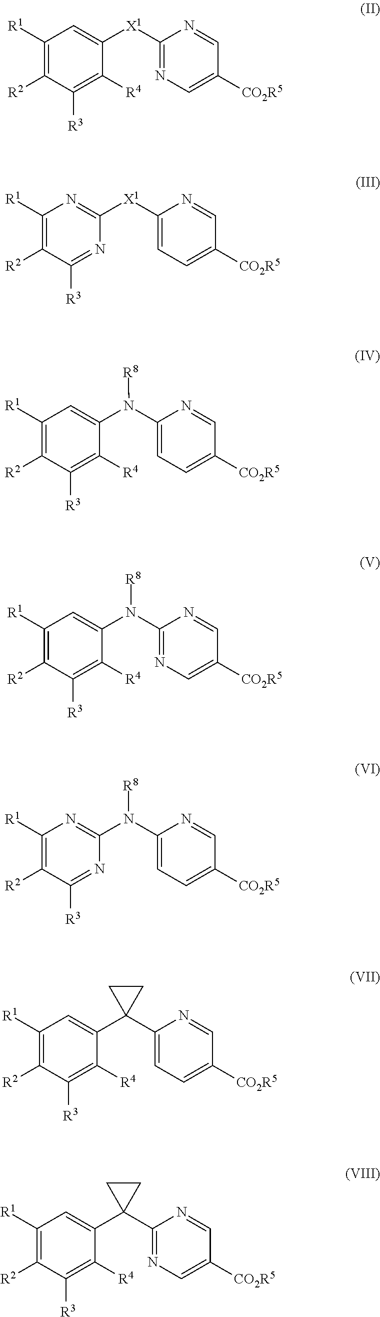

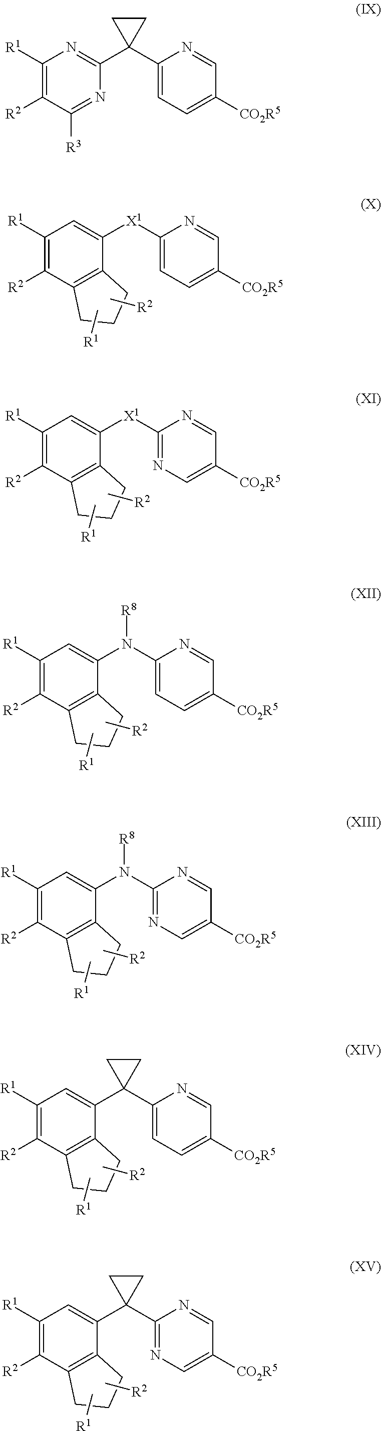

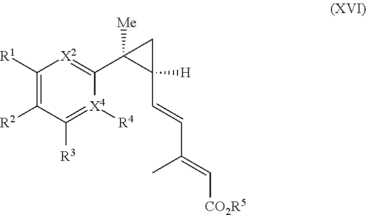

or a pharmaceutically acceptable salt thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl group;
each X² is, independently, N or CR⁹, wherein R⁹ is H or alkyl or R⁸ and R⁹, together with the atoms to which they are each attached, form a heterocyclyl group;
X³ is CH or N;
X⁴ is N or C;
R¹ is alkyl, or R¹ and R² together can form a ring;
R² is H, alkyl, alkoxy, or R¹ and R² together can form a ring, provided in some cases that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group.

Embodiment 5 relates to the method of Embodiments 1-4, wherein the RXR agonist is a compound of the formula:

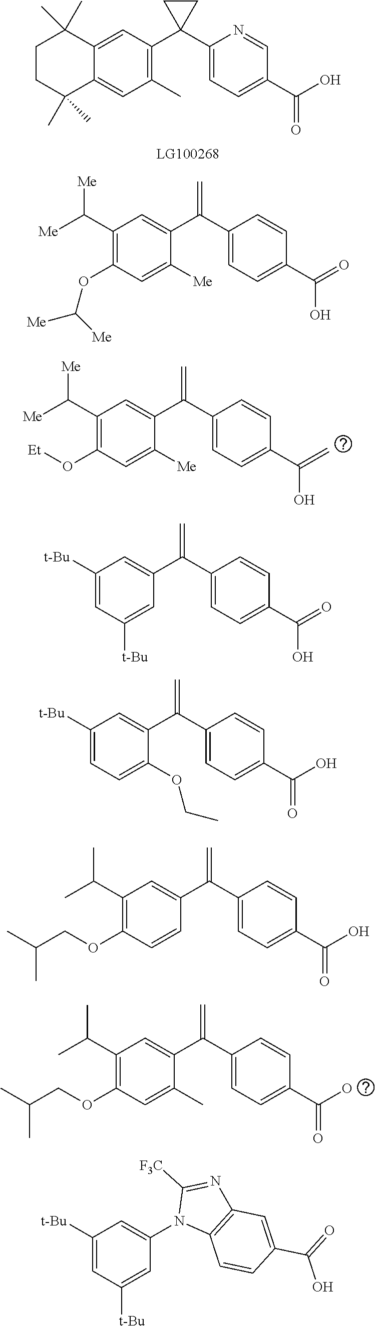

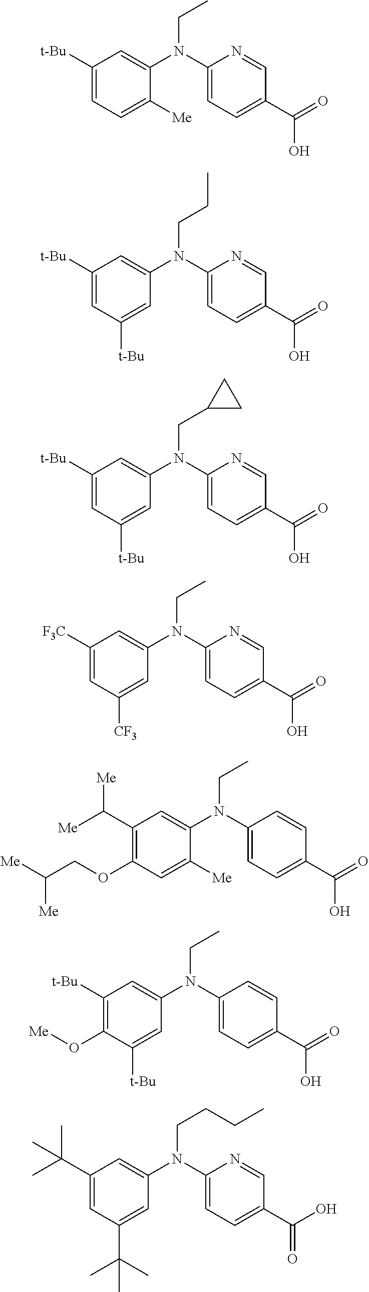

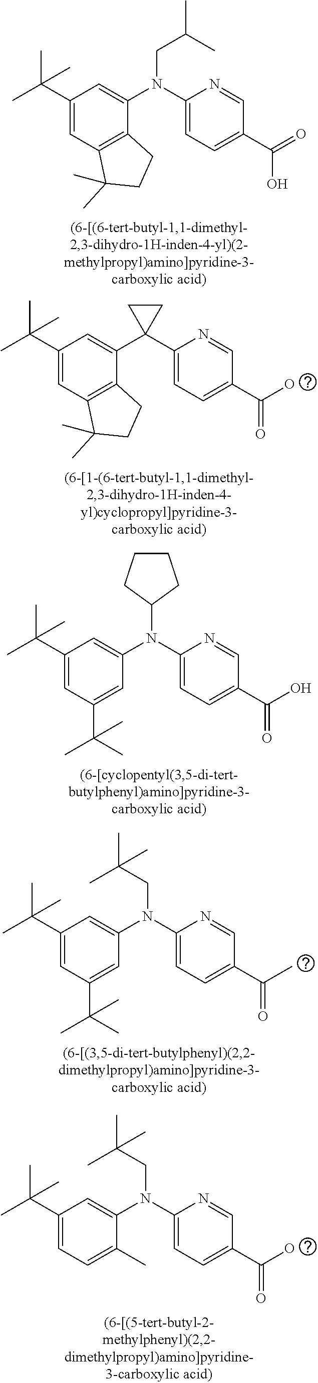

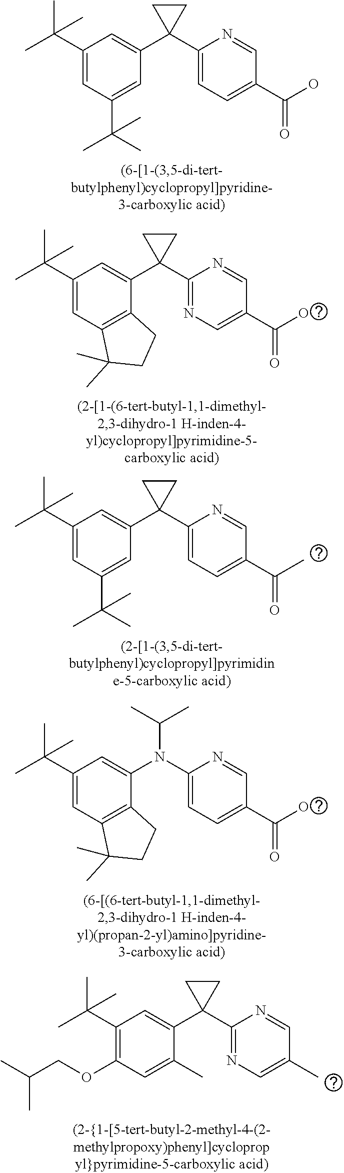

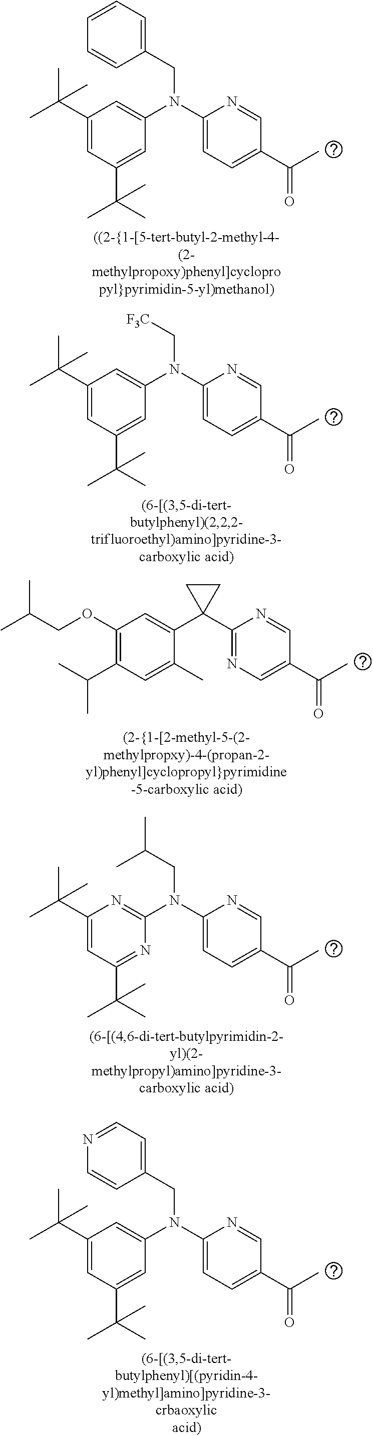

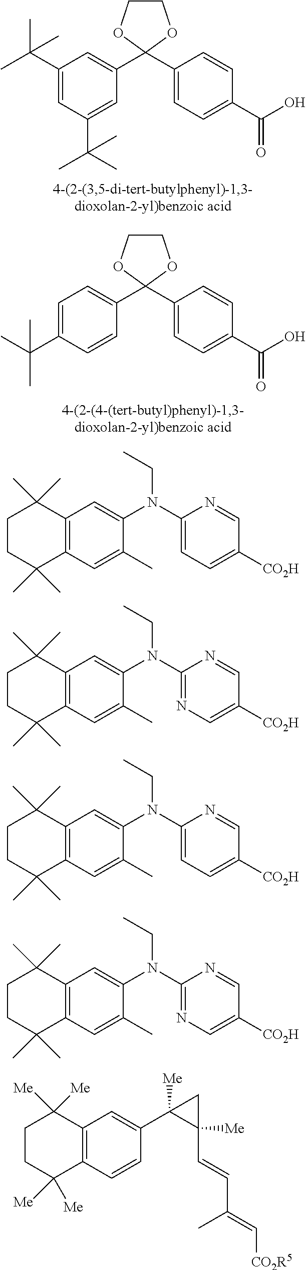

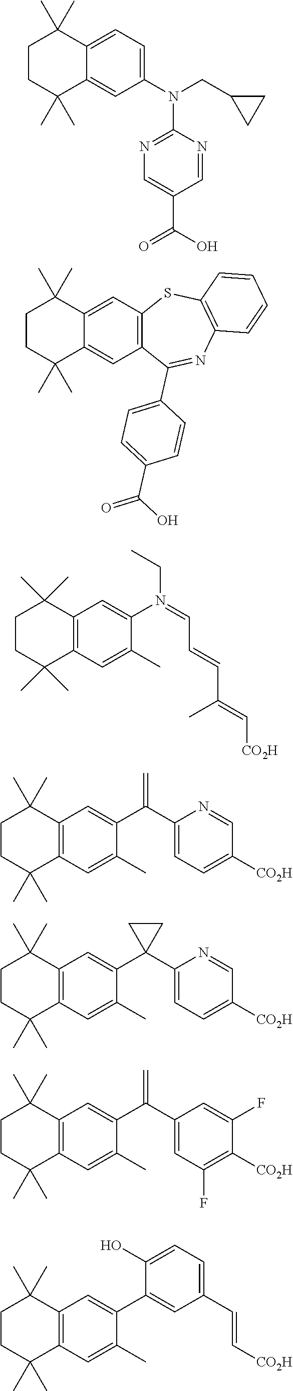

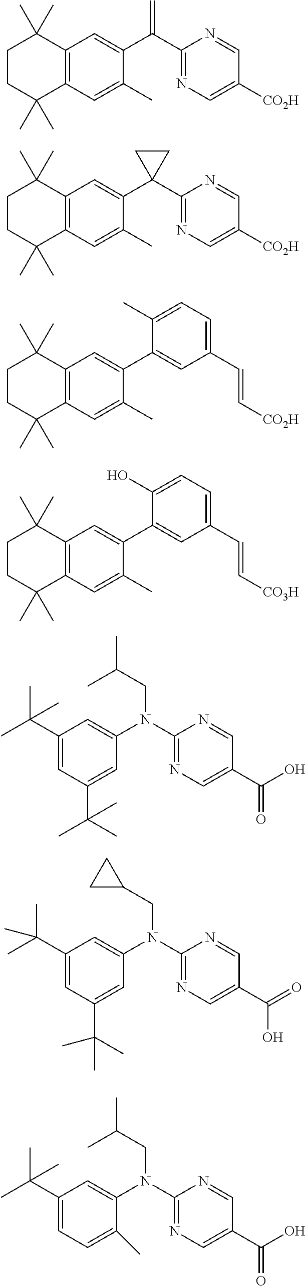

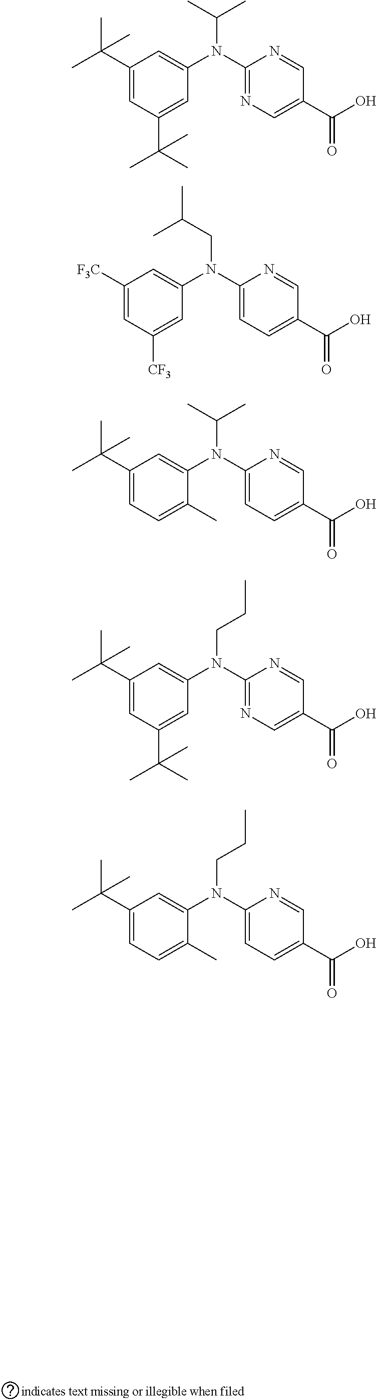

or a combination thereof.

Embodiment 6 relates to the compound of Embodiments 1-5, wherein the RXR agonist is a compound of Formula (I):

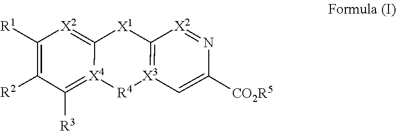

or a pharmaceutically acceptable salt thereof, wherein:
X¹ is NR⁸, wherein R⁸ is H or alkyl;
each X² is, independently, N or CR⁹, wherein R⁹ is H or alkyl;
X³ is CH or N;
X⁴ is N or C;
R¹ is alkyl;
R² is H, alkyl or alkoxy, optionally provided that when X² is N and X³ is CH, then
R² is not isobutoxy;
R³ is H or alkyl;
R⁴ is absent, H, alkyl or alkoxy; and
R⁵ is H or alkyl;
or R² and R³ or R³ and R⁴, together with the carbon atoms to which they are attached, form a cycloalkyl group;
wherein the compound of Formula (I) is optionally at least substituted at two of R¹, R², R³, and R⁴.

Embodiment 7 relates to the method of Embodiment 6, wherein R² and R³, together with the carbon atoms to which they are attached, form a cycloalkyl group.

Embodiment 8 relates to the method of Embodiment 6, wherein R³ and R⁴, together with the carbon atoms to which they are attached, form a cycloalkyl group.

Embodiment 9 relates to the method of Embodiment 6, wherein R¹ and R³ can each, independently be, alkyl.

Embodiment 10 relates to the method of Embodiment 6, wherein R¹ is alkyl and R² is H or alkoxy.

Embodiment 11 relates to the method of Embodiment 6, wherein R¹ and R³ are each, independently, C₁-C₆-alkyl groups

Embodiment 12 relates to the method of Embodiment 11, wherein R¹ and R³ are each t-butyl groups.

Embodiment 13 relates to the method of Embodiments 6-12, wherein R⁹ is H.

Embodiment 14 relates to the method of Embodiments 6-13, wherein X² is N and X³ is CH.

Embodiment 15 relates to the method of Embodiments 6-13, wherein X² is CH and X³ is CH.

Embodiment 16 relates to the method of Embodiments 6-15, wherein R⁴ is H.

Embodiment 17 relates to the method of Embodiments 6-15, wherein R¹ is alkyl and R⁴ is alkoxy.

Embodiment 18 relates to the method of Embodiments 6-17, wherein R⁵ is H.

Embodiment 19 relates to the method of Embodiment 6-18, wherein the compound of the Formula (I) is a compound of the Formula (II)-(VII):

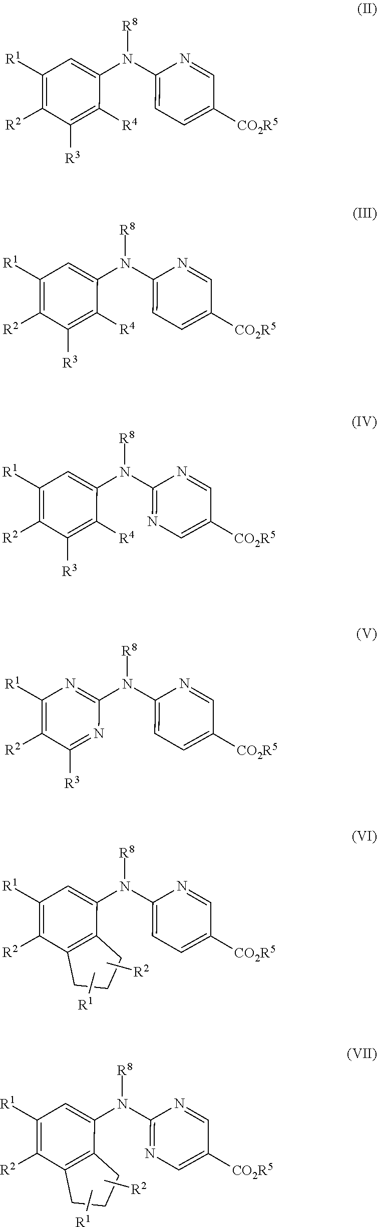

or a pharmaceutically acceptable salt thereof.

Embodiment 20 relates to the method of Embodiments 1-19, wherein the compound of the Formula (I) is a compound of the formula:

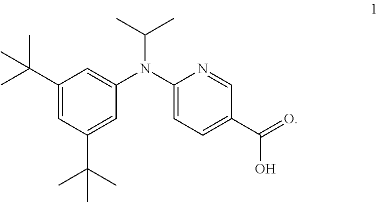 [SMILES: CC(C)N(c1cc(C(C)(C)C)cc(C(C)(C)C)c1)c1ccc(C(=O)O)cn1]

or a pharmaceutically acceptable salt thereof.

Embodiment 21 relates to the method of Embodiments 1-20, wherein the RAS inhibitor is AMG-510, MRTX 849, captopril, imidapril, zofenopril, candesartan, delapril, telmisartan, aliskiren, moexipril, enalapril, valsartan, fosinopril, irbesartan, perindopril, quinapril, ramipril, eprosartan, olmesartan, trandolapril, losartan, azilsartan, lisinopril, spirapril, benazepril or cilazapril or pharmaceutically acceptable salts thereof.

Embodiment 22 relates to the method of Embodiments 1-21, wherein the MEK inhibitor is refametinib, selumetinib, trametinib, cobimetinib, pimasertib, RO-4987655, RO5126766, WX-554, HL-085, PD-0325901, MEK 162, AZD-8330, TAK-733 or GDC-0623 or pharmaceutically acceptable salts thereof.

Embodiment 23 relates to the method of Embodiments 1-22, wherein the BRAF inhibitor is vemurafenib, dabrafenib, encorafenib or binimetinib or pharmaceutically acceptable salts thereof.

Embodiment 24 relates to a method for treating plexiform neurofibromas (PNFs) or malignant peripheral nerve sheath tumors (MPNSTs), the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

Embodiment 25 relates to a method of treating RASopathies, the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

Embodiment 26 relates to the method of Embodiment 25, wherein the RASoptathies are neurofibromatosis type 1, Noonan syndrome, Noonan syndrome with multiple lentigines, capillary malformation-arteriovenous malformation syndrome, Costello syndrome, cardio-facio-cutaneous syndrome or Legius syndrome or a combination thereof.

Embodiment 27 relates to a method of treating schwannomas or gliomas, the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

Embodiment 28 relates to the method of Embodiment 27, wherein the glioma is an optical pathway glioma, a low-grade glioma, and/or a high-grade glioma.

Embodiment 29 relates to a method of treating cancer, the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

Embodiment 30 relates to the method of Embodiment 29, wherein the cancer is rhabdomyosarcoma, neuroblastoma, juvenile myelomonocytic leukemia, gastronintestinal stromal tumors, carcinoid, breast cancer, endocrine cancer, melanoma, acute lymphoblastic leukemia, ovarian cancer, prostate cancer, meningioma, and undifferentiated pleomorphic sarcoma.

Embodiment 31 relates to the method of Embodiment 30, wherein the endocrine cancer is pheochromocytoma or a neuroendocrine tumor.

The following non-limiting Examples illustrate some aspects of the development of the invention.

### EXAMPLES

The disclosure can be better understood by reference to the following examples which are offered by way of illustration. The disclosure is not limited to the examples given herein.

The compounds described herein can be made using a variety of methods. Exemplary methods are illustrated below in Schemes 1 and 2.

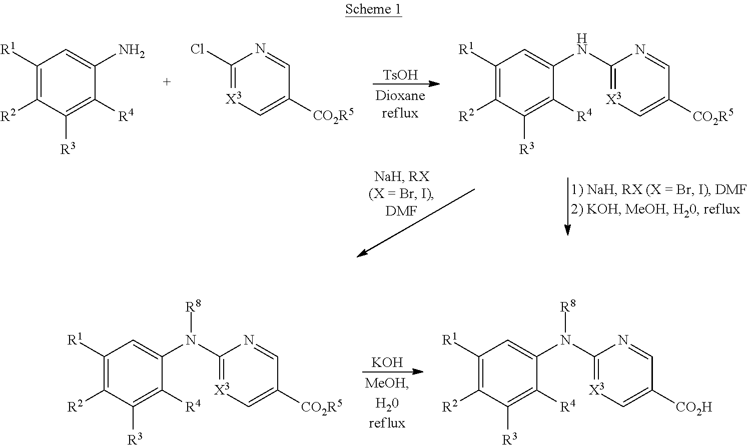

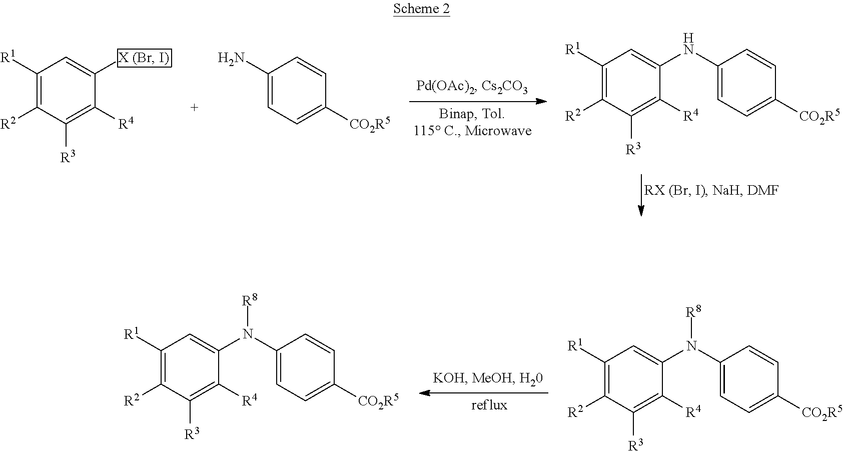

### Compound 1, Also Known As 6-[(3,5-di-tert-butylphenyl) (propan-2-yl)amino]pyridine-3-carboxylic acid, Was Specifically Synthesized as Follows

A solution of 4-[(3,5-di-tert-butyl-4-methoxyphenyl) (ethyl)amino]-benzoate (0.13 g, 0.37 mmol) in dimethylformamide (5.0 mL) was treated with 60% sodium hydride (0.15 g, 3.6 mmol), stirred for 15 minutes, treated with 2-iodopropropane (0.62 g, 3.6 mmol) and the mixture stirred at 50° C. for 14 hours. The mixture was then quenched with an aqueous solution of saturated ammonium chloride and extracted with ethyl acetate. The organic layers were combined, dried with sodium sulfate, filtered and the solvent removed in vacuo. The residue was then purified by MPLC (SiO₂, 100% dichloromethane gradient to 5% methanol/dichloromethane) to provide the acid as an oil (0.054 g, 40%). 1H NMR (500 MHZ, DMSO-d6) δ 8.65 (dd, J=2.4, 0.8 Hz, 1H), 7.72 (dd, J=9.1, 2.4 Hz, 1H), 7.43 (t, J=1.8 Hz, 1H), 6.96 (d, J=1.8 Hz, 2H), 5.84 (dd, J=9.0, 0.8 Hz, 1H), 5.29 (hept, J=6.7 Hz, 1H), 1.28 (s, 18H), 1.04 (d, J=6.7 Hz, 6H).

### Example 1: Compound Identification

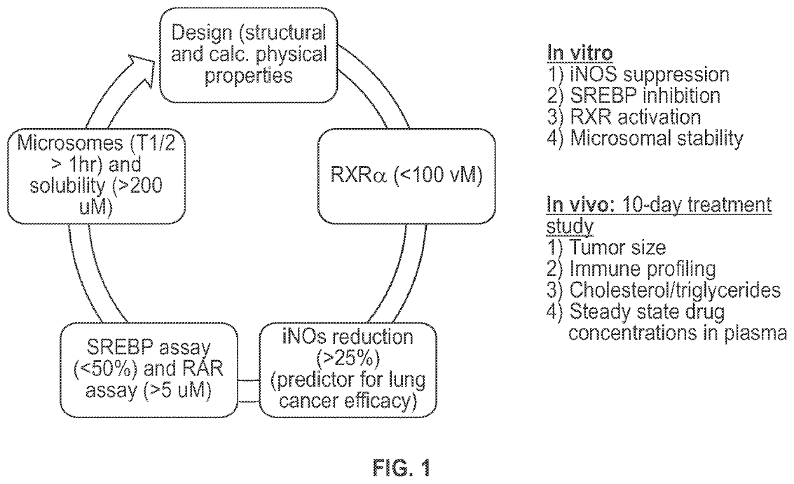

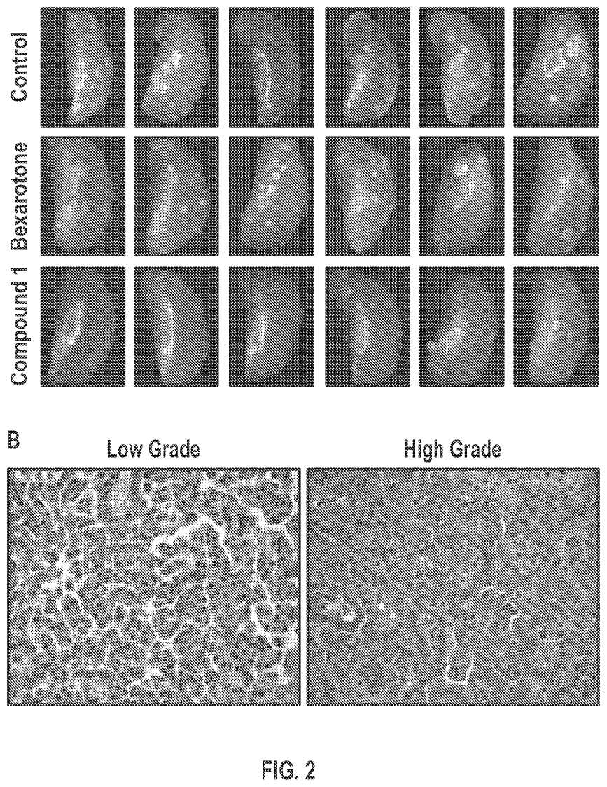

Compound 1 was identified using an optimized screening funnel (FIG. 1), Compound 1 is more potent than bexarotene in activating RXR; the EC50 is 30 nM for compound 1 vs. 55 nM for bexarotene. In contrast to bexarotene, compound 1 does not elevate triglycerides in vivo. Notably, compound 1 has better single agent activity than bexarotene for treating established Kras-driven lung adenocarcinomas. Compared to both untreated controls and mice treated with bexarotene, compound 1 significantly (p<0.05) decreased the number, size and histopathology of lung tumors (FIG. 2). The average tumor burden was 60% lower (p=0.015) in the lungs of mice treated with compound 1 compared to the control and bexarotene groups. The histopathology of the tumors was also improved with compound 1 treatment, as 77% of the tumors in the control group were high grade vs. only 62% in the compound 1 group (p<0.05).

### Example 2: Immune Modulation

Instead of directly targeting Ras, compound 1 favorably modulates inflammatory mediators and immune cell populations known to be regulated by Kras mutations or Ras pathway activation. Crosstalk between the tumor cells and CD206+ macrophages, myeloid derived suppressor cells (MDSCs), and regulatory T cells (Tregs) suppress anti-tumor immunity and promote tumor growth. It has been previously shown that the tool RXR agonist LG100268 not only decreased CD206+ tumor-promoting macrophages, immunosuppressive MDSCs, and immunosuppressive Foxp3+ Tregs but also increased the number of activated cytotoxic CD8 T cells in tumors of MMTV-neu mice, in which the Ras pathway is activated. These changes are all highly beneficial for treating cancer. Notably, these changes in the immune system were not observed with bexarotene, the FDA-approved RXR agonist. Compound 1 alters the same immune cells in this model as LG100268. Despite similarities (RAS activation and immune cell infiltration that drives pathogenesis) in the biology of NF1 and lung cancer, no RXR agonist has ever been tested for NF1.

### Example 3: Compound 1 Inhibits Tumor Growth and p-ERK Levels and Alters Immune Cell Populations

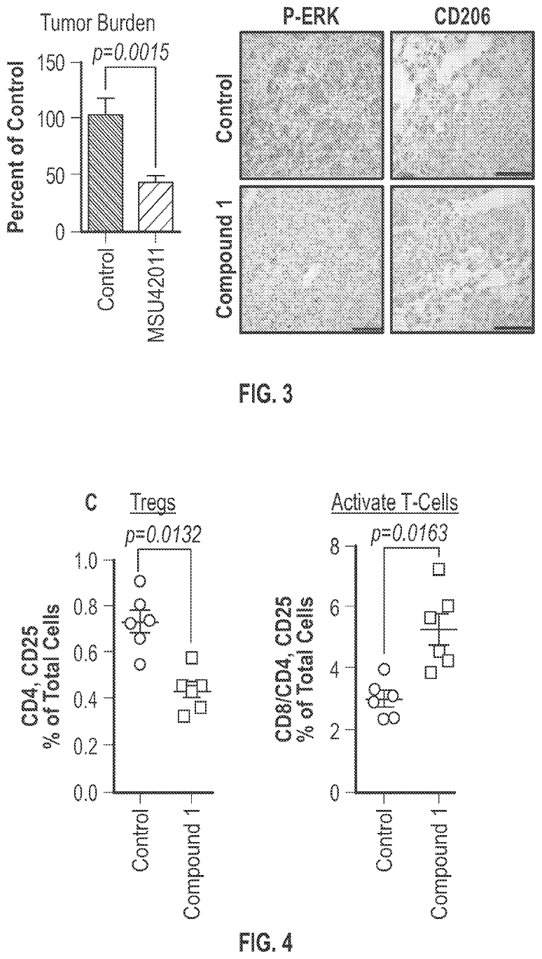

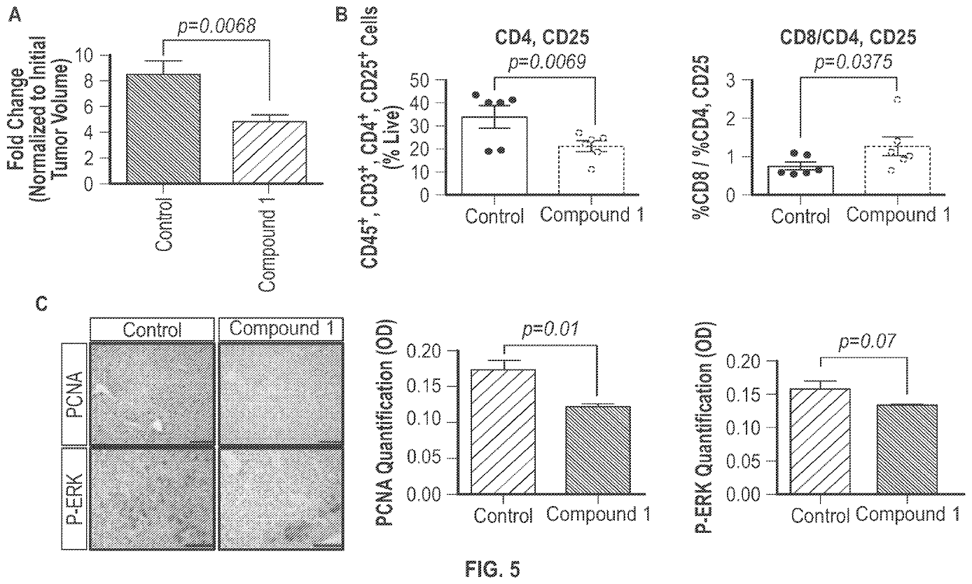

Compound 1 inhibits tumor growth and p-ERK levels and alters immune cell populations in RAS-driven tumor models. When used to treat established lung tumors with an activating Kras mutation, compound 1 significantly (p=0.0015) reduced lung tumor burden (FIG. 3). Compound 1 also reduced p-ERK levels (p=0.03) and tumor-promoting CD206+ macrophages within the lung tumors (FIG. 3). As shown by flow cytometry (FIG. 4), Compound 1 significantly (p<0.05) decreased immunosuppressive CD4+CD25+ Tregs and increased activated cytotoxic T cells (ratio of CD8+/CD4+, CD25+) that kill cancer cells; these results were confirmed by IHC. Similar results were observed in the MMTV-neu model of breast cancer, in which the Ras pathway is activated (FIG. 5).

### Example 4: Combination with Selumetinib

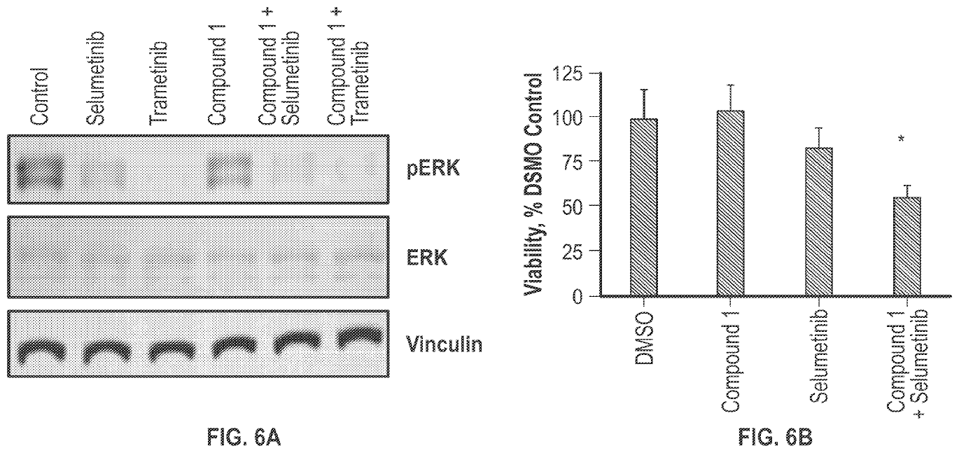

Pilot studies showed that compound 1 in combination with selumetinib reduces p-ERK expression by approximately 70%, 40% and 90%, respectively, in human NF1 deficient plexiform neurofibroma cells (FIG. 6A). p-ERK is elevated in these human cells because they contain both a germline and somatic mutation in NF1, which activates RAS and the downstream MAPK signaling pathway. The combination of drugs also inhibits viability of these cells more effectively than each individual drug (FIG. 6B).

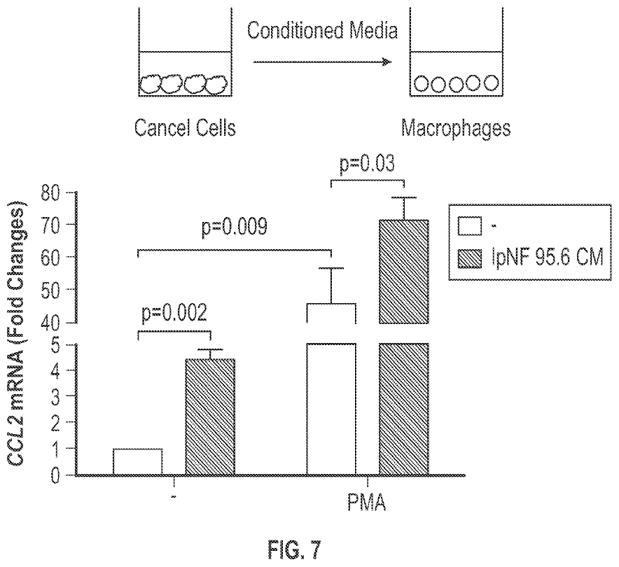

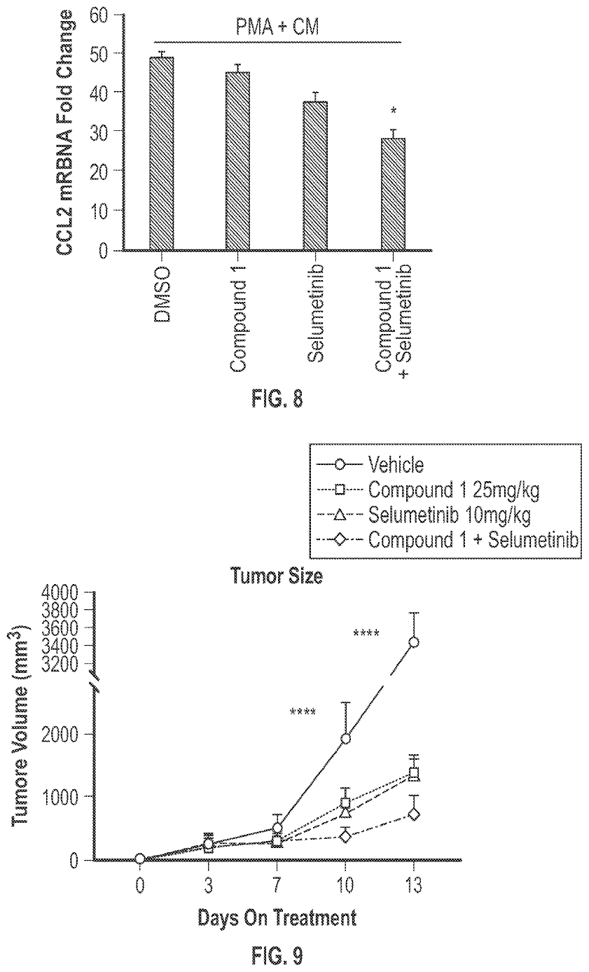

NF1 deficiency in mice led to increased infiltration of macrophages in tumors. The extent of macrophage recruitment into both mouse and human tumors increased with disease severity, from normal nerve to nerve with NF1 mutation to neurofibromas to malignant peripheral nerve sheath tumors. Initial studies in NF1-deficient human neurofibroma (ipNF95.6) cells showed that conditioned media (CM) from human THP-1 monocytes differentiated into macrophages with phorbol 12-myristate 13-acetate (PMA) increases p-ERK expression 2.5 fold in these plexiform neurofibroma cells. Conversely, CM from the ipNF95.6 cells significantly increased CCL2 mRNA expression in THP-1 cells, and PMA differentiation of THP-1 cells into macrophages enhances this increase in CCL2 (FIG. 7). These studies are important as the chemokine CCL2 not only recruits macrophages to sites of inflammation or injury but also correlates with disease status and growth of neurofibromas. As shown in FIG. 8, compound 1 modestly decreased CCL2 mRNA expression by 23%, but the combination of drugs reduced CCL2 mRNA by 42% (p<0.05).

Additional studies were conducted where 1×10⁶ LL2 cells were implanted subcutaneously into the flank of 7-8 week old male C57/B16 mice. When tumors reached ˜3×3 mm mice were randomized into treatment groups (vehicle, compound 1, 25 mg/kg, selumetinib 10 mg/kg, or a combination of compound 1 and selumetinib). Treatment was given 5 times per week for two weeks and tumors measured on Mondays and Thursdays using calipers (n=8 per group). The data collected is shown in FIGS. 9A and 9B.

### Example 5: CCI2 mRNA Expression Studies

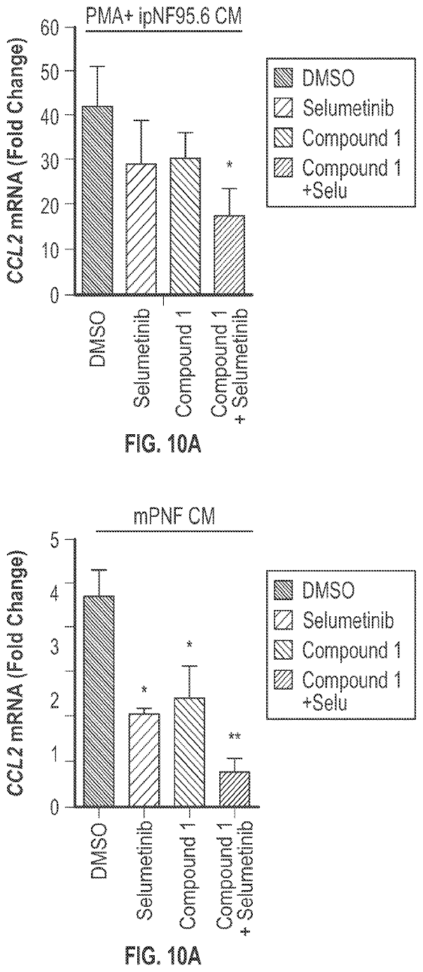

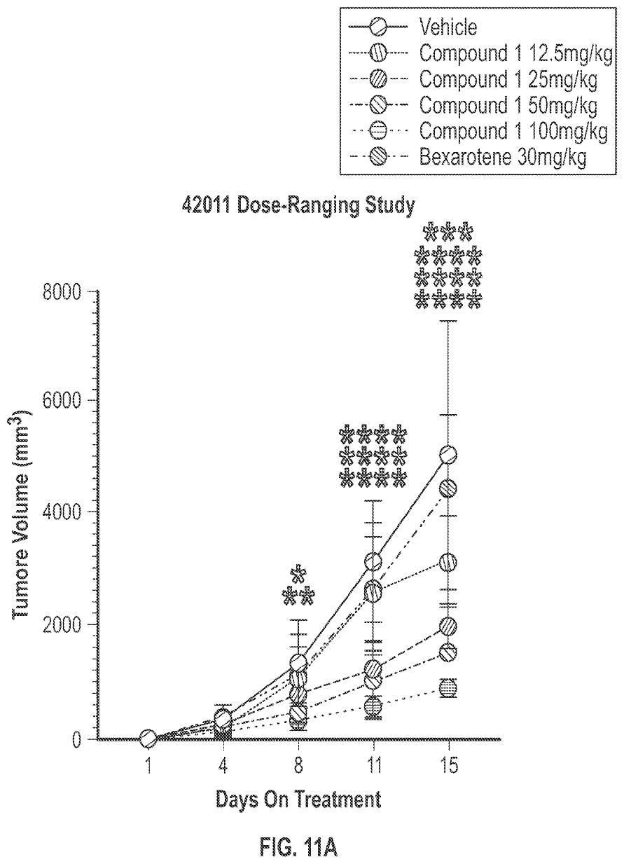

Combination treatment with compound 1 and selumetinib decreased CCL2 mRNA expression in THP1 cells and BMDM stimulated with conditioned media (CM) from human and mouse PNF cells. FIG. 10A shows the results where THP1 monocytes were differentiated into macrophages with 50 ng/ml PMA for 3 days, followed by treatment with CM from human PNF cells (ipNF95.6) and drugs (50 nM selumetinib, 200 nM compound 1, or the combination) for an additional 24 hrs. FIG. 10B shows the results where BMDM were differentiated into macrophages with 20 ng/ml M-CSF for 5 days, followed by treatment with CM from mouse PNF cells (NF1f/f Cre+) and drugs (50 nM selumetinib, 200 nM compound 1, or the combination) for an additional 24 hrs. CCL2 mRNA expression was evaluated by qPCR and normalized to the DMSO control without CM treatment (not shown). Data represent means±standard deviations. * p<0.05, ** p<0.01 compared with vehicle treatment.

### Example 6: Compound 1 and Beraxotene Dose Ranging Studies

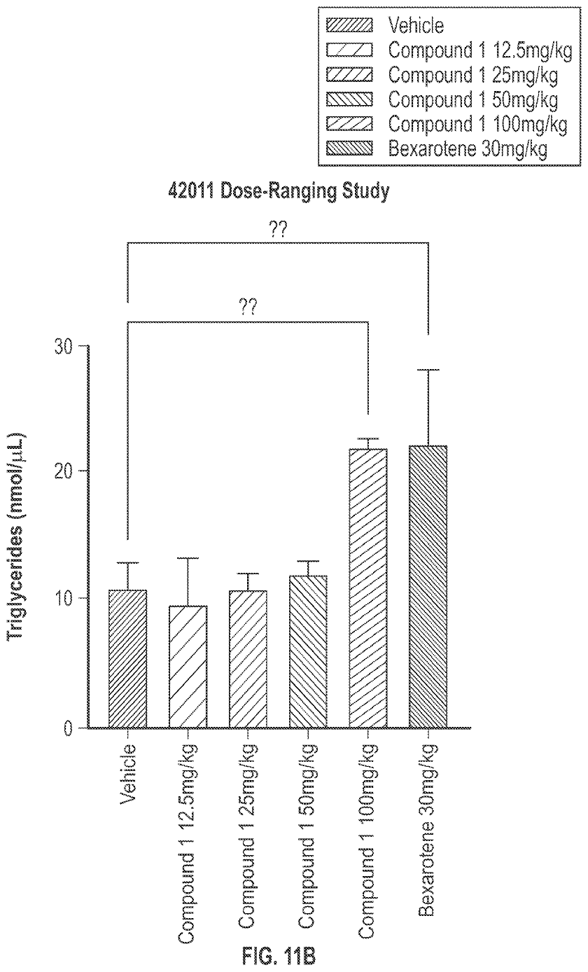

In this study, 7-8 week old male C57/BL6 mice were injected with 1×10⁶ LL2 cells subcutaneously into the left flank. Tumors were measured with calipers twice per week (Monday and Thursday) and mice were weighed once per week (Monday). When tumors reached 3×3 mm, mice were randomized into treatment groups (n=8) and treated with either vehicle control (DMSO:Cremaphor:Saline 1:1:8), compound 1 (12.5-100 mg/kg), or bexarotene (30 mg/kg). When tumors reached endpoint (20×20 mm or ulceration), mice were euthanized and tumors fixed in formalin. Blood was collected for plasma. Stats: two-way ANOVA w/Tukey HSD. The data are shown in FIG. 11A. The data in FIG. 11B show triglyceride levels measured under the same conditions as for the data in FIG. 11A.

1. A method for treating NF1 or conditions resulting from NF1 function loss the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

2. The method of claim 1, wherein the RXR agonist can be formulated together or separately with at least one inhibitor of RAS, MEK, and BRAF or administered together or separately with at least one inhibitor of RAS, MEK, and BRAF.

3. The method of claim 1, wherein the RXR agonist is a compound of the Formula (I): 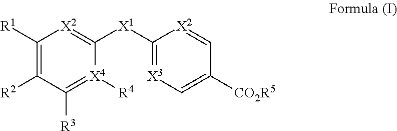
or a pharmaceutically acceptable salt, polymorph, prodrug, solvate or clathrate thereof, wherein:
X¹ is C═C(R⁶R⁷), CRÓR⁷ or NR⁸, wherein R⁶-R⁸ are each independently H or alkyl or the R⁶ and R⁷ groups on CR⁶R⁷, together with the carbon atom to which they are attached, form a cycloalkyl or heterocyclyl group;
each X² is, independently, N or CR⁹, wherein R⁹ is H or alkyl or R⁸ and R⁹, together with the atoms to which they are each attached, form a heterocyclyl group;
X³ is CH or N;
X⁴ is N or C;
R¹ is alkyl;
R² is H, alkyl or alkoxy, provided in some cases that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group.

4. The method of claim 1, wherein the RXR agonist is a compound of the formula: 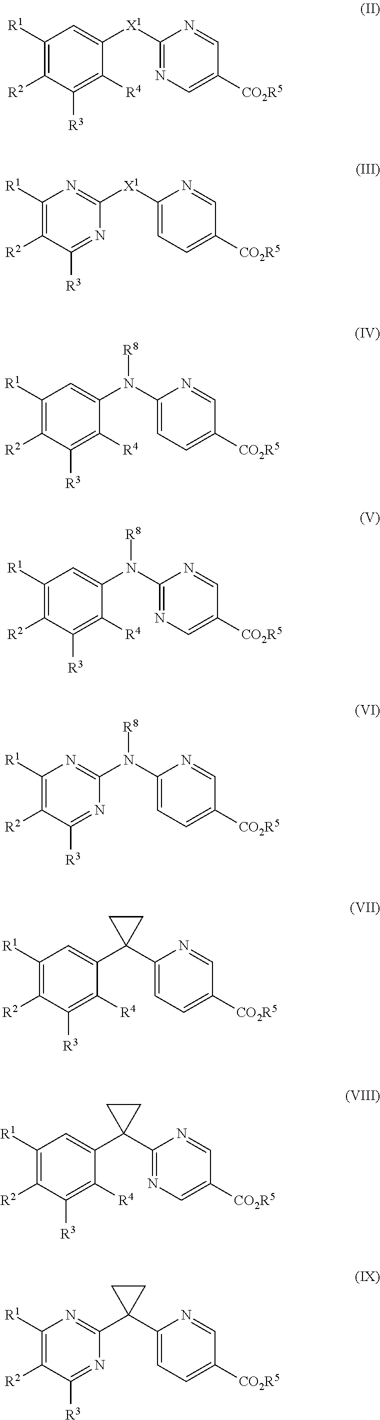 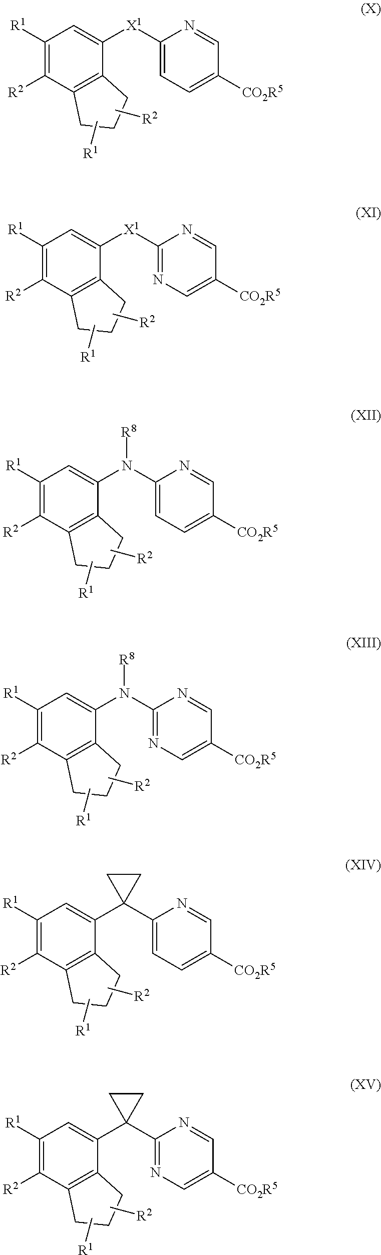 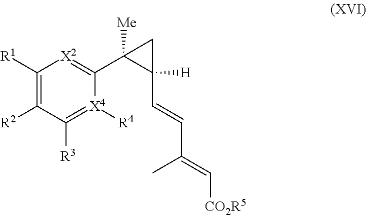
or a pharmaceutically acceptable salt thereof, wherein:
X¹ is C═C(R⁶R⁷), CR⁶R⁷ or NR⁸, wherein R⁶-R⁸ are each independently H or alkyl or the R⁶ and R⁷ groups on CR⁶R⁷, together with the carbon atom to which they are attached, form a cycloalkyl group;
each X² is, independently, N or CR⁹, wherein R⁹ is H or alkyl or R⁸ and R⁹, together with the atoms to which they are each attached, form a heterocyclyl group,
X³ is CH or N;
X⁴ is N or C;
R¹ is alkyl, or R¹ and R² together can form a ring;
R² is H, alkyl, alkoxy, or R¹ and R² together can form a ring, provided in some cases that when X² is N and X³ is CH, then R² is not isobutoxy;
R⁴ is absent, H, alkyl or alkoxy;
R³ is H or alkyl; and
R⁵ is H or alkyl;
or R² and R³ or R³ and R⁴, together with the carbon atoms to which they are attached, form a cycloalkyl group.

5. The method of claim 1, wherein the RXR agonist is a compound of the formula: 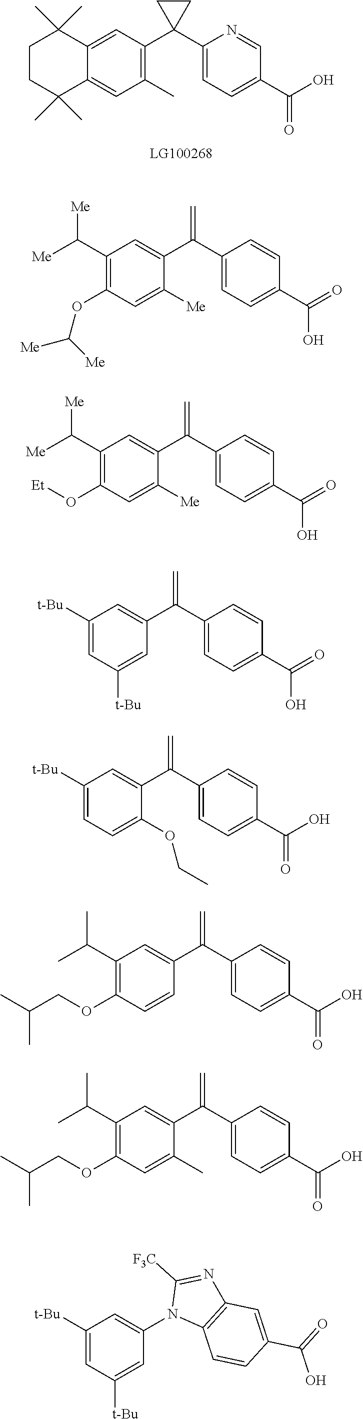 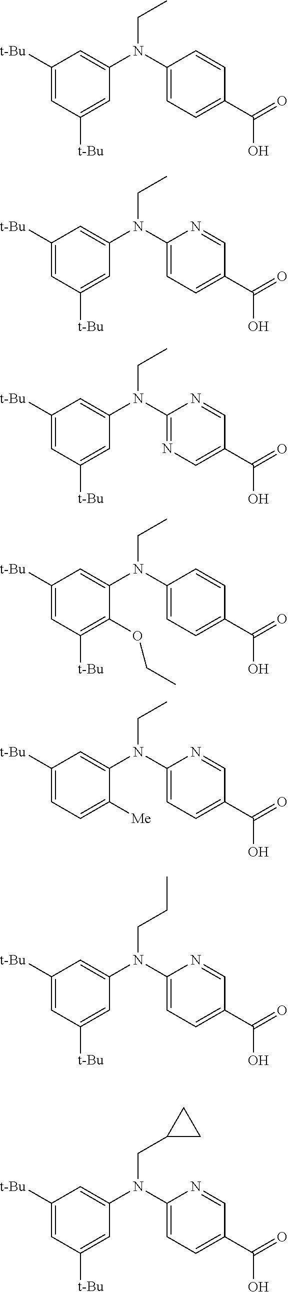 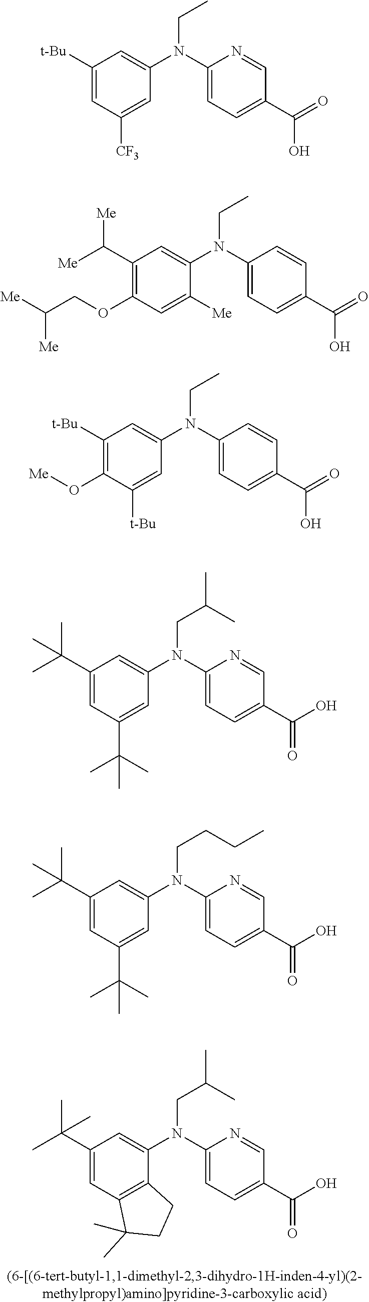 [SMILES: CCN(c1cc(C(C)(C)C)cc(C(F)(F)F)c1)c1ccc(C(=O)O)cn1 ; CCN(c1ccc(C(=O)O)cc1)c1cc(C(C)C)c(OCC(C)C)cc1C ; CCN(c1ccc(C(=O)O)cc1)c1cc(C(C)(C)C)c(OC)c(C(C)(C)C)c1 ; CC(C)CN(c1cc(C(C)(C)C)cc(C(C)(C)C)c1)c1ccc(C(=O)O)cn1 ; CCCCN(c1cc(C(C)(C)C)cc(C(C)(C)C)c1)c1ccc(C(=O)O)cn1 ; CC(C)CN(c1ccc(C(=O)O)cn1)c1cc(C(C)(C)C)cc2c1CCC2(C)C] 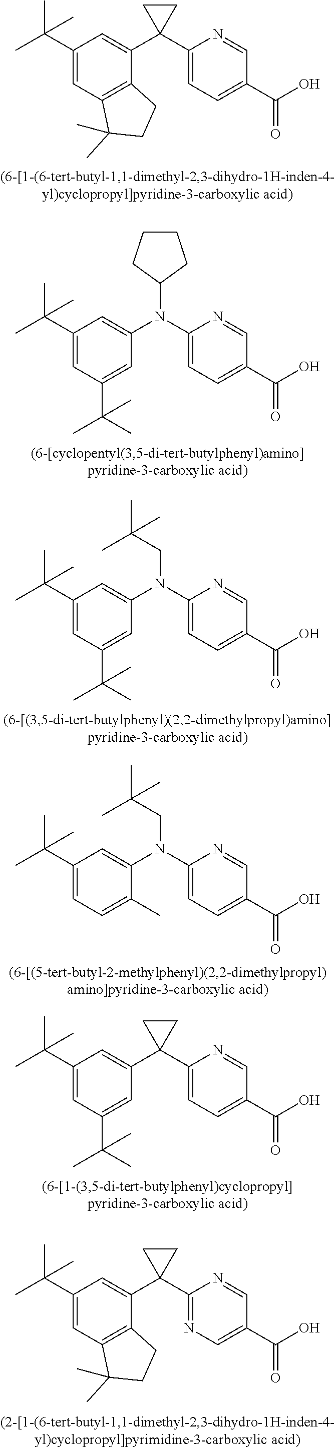 [SMILES: CC(C)(C)c1cc2c(c(C3(c4ccc(C(=O)O)cn4)CC3)c1)CCC2(C)C ; CC(C)(C)c1cc(N(c2ccc(C(=O)O)cn2)C2CCCC2)cc(C(C)(C)C)c1 ; CC(C)(C)CN(c1cc(C(C)(C)C)cc(C(C)(C)C)c1)c1ccc(C(=O)O)cn1 ; Cc1ccc(C(C)(C)C)cc1N(CC(C)(C)C)c1ccc(C(=O)O)cn1 ; CC(C)(C)c1cc(C(C)(C)C)cc(C2(c3ccc(C(=O)O)cn3)CC2)c1 ; CC(C)(C)c1cc2c(c(C3(c4ncc(C(=O)O)cn4)CC3)c1)CCC2(C)C] 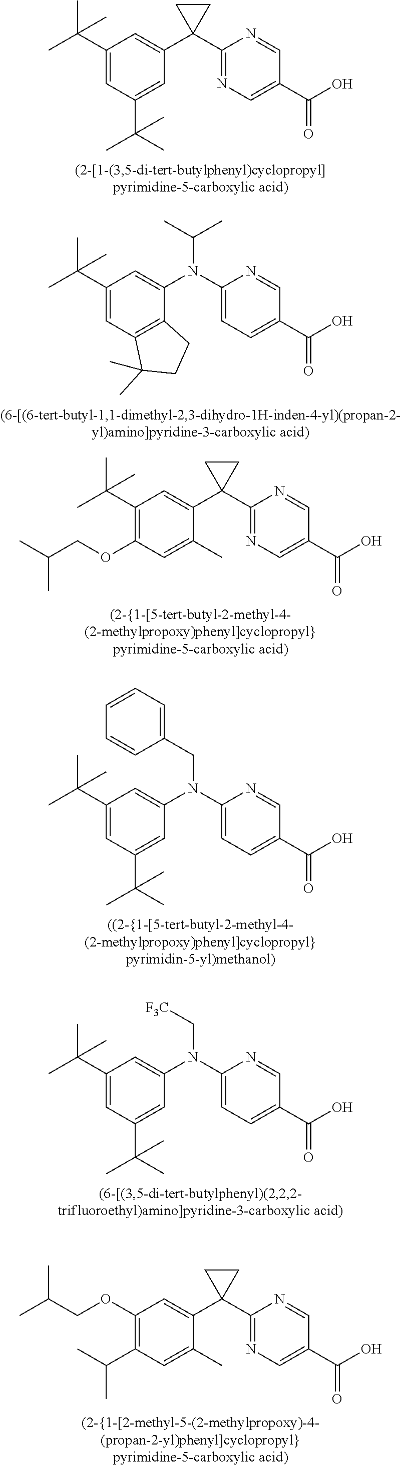 [SMILES: CC(C)(C)c1cc(C(C)(C)C)cc(C2(c3ncc(C(=O)O)cn3)CC2)c1 ; CC(C)N(c1ccc(C(=O)O)cn1)c1cc(C(C)(C)C)cc2c1CCC2(C)C ; Cc1cc(OCC(C)C)c(C(C)(C)C)cc1C1(c2ncc(C(=O)O)cn2)CC1 ; CC(C)(C)c1cc(N(Cc2ccccc2)c2ccc(C(=O)O)cn2)cc(C(C)(C)C)c1 ; CC(C)(C)c1cc(N(CC(F)(F)F)c2ccc(C(=O)O)cn2)cc(C(C)(C)C)c1 ; Cc1cc(C(C)C)c(OCC(C)C)cc1C1(c2ncc(C(=O)O)cn2)CC1] 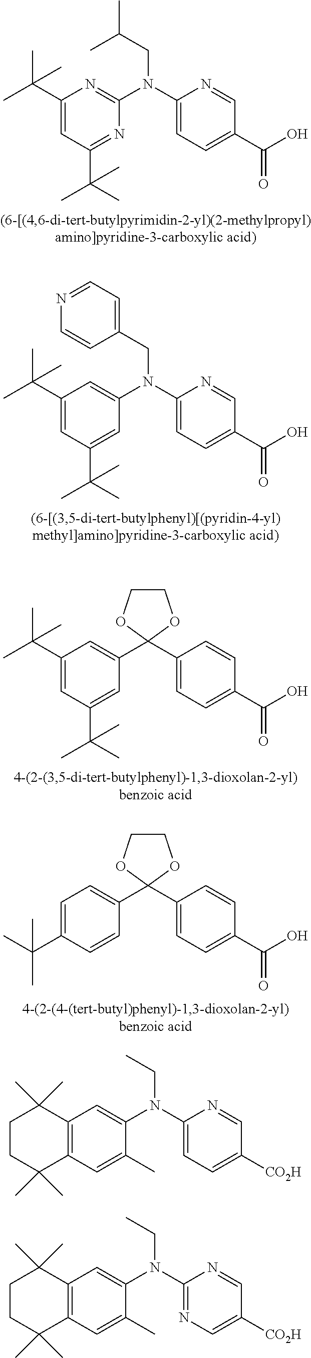 [SMILES: CC(C)CN(c1ccc(C(=O)O)cn1)c1nc(C(C)(C)C)cc(C(C)(C)C)n1 ; CC(C)(C)c1cc(N(Cc2ccncc2)c2ccc(C(=O)O)cn2)cc(C(C)(C)C)c1 ; CC(C)(C)c1cc(C(C)(C)C)cc(C2(c3ccc(C(=O)O)cc3)OCCO2)c1 ; CC(C)(C)c1ccc(C2(c3ccc(C(=O)O)cc3)OCCO2)cc1 ; CCN(c1ccc(C(=O)O)cn1)c1cc2c(cc1C)C(C)(C)CCC2(C)C ; CCN(c1ncc(C(=O)O)cn1)c1cc2c(cc1C)C(C)(C)CCC2(C)C] 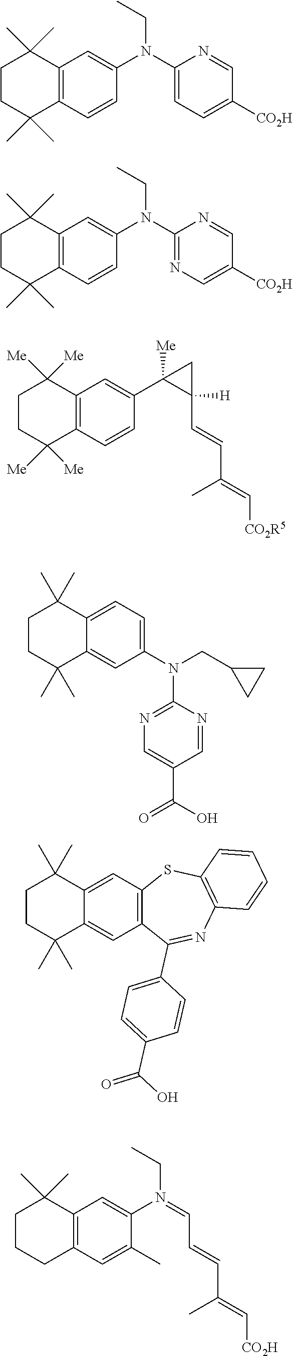 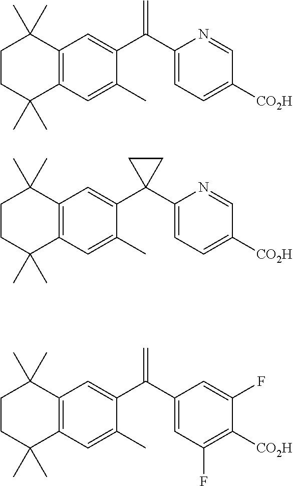 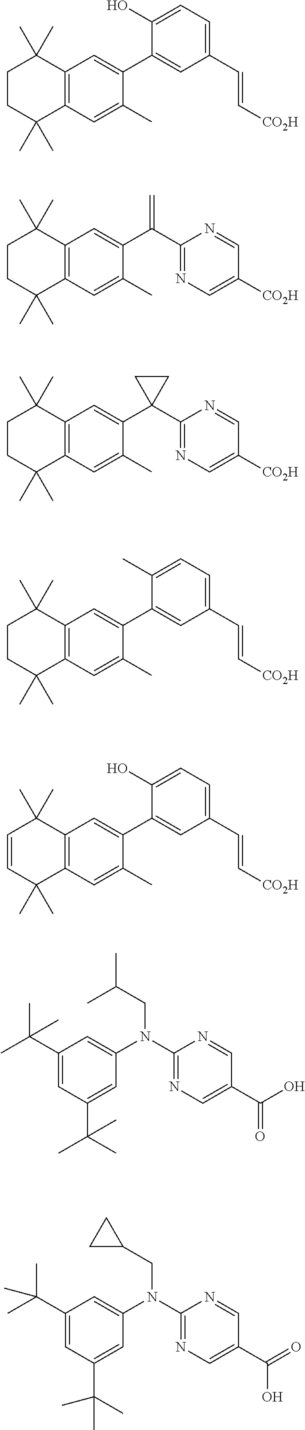 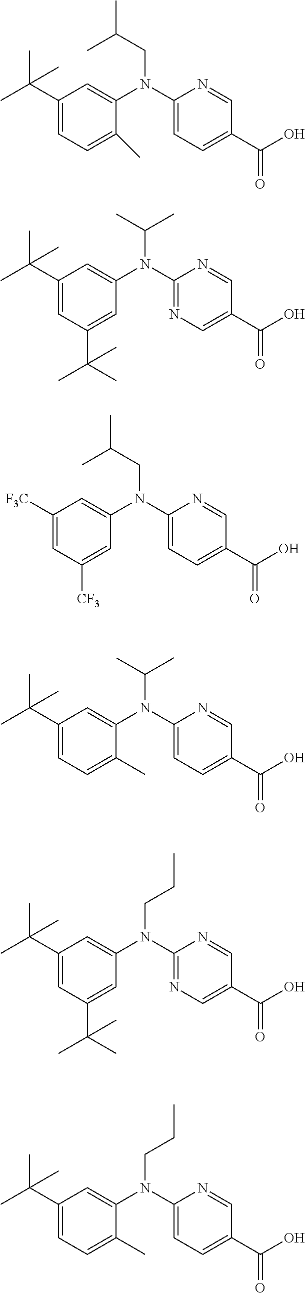
or a combination thereof.

6. The method of claim 1, wherein the RXR agonist is a compound of Formula (I): 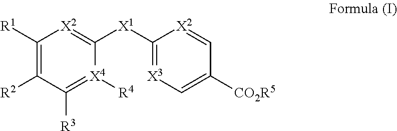
or a pharmaceutically acceptable salt thereof, wherein:
X¹ is NR⁸, wherein R⁸ is H or alkyl;
each X² is, independently, N or CR⁹, wherein R⁹ is H or alkyl;
X³ is CH or N;
X⁴ is N or C;
R¹ is alkyl;
R² is H, alkyl or alkoxy, provided that when X² is N and X³ is CH, then R² is not isobutoxy;
R³ is H or alkyl;
R⁴ is absent, H, alkyl or alkoxy; and
R⁵ is H or alkyl;
or R² and R³ or R³ and R⁴, together with the carbon atoms to which they are attached, form a cycloalkyl group;
wherein the compound of Formula (I) is at least substituted at two of R¹, R², R³, and R⁴.

7. The method of claim 6, wherein;
R² and R³, together with the carbon atoms to which they are attached, form a cycloalkyl group;
R³ and R⁴, together with the carbon atoms to which they are attached, form a cycloalkyl group;
R¹ and R³ are each, independently, alkyl:
R¹ is alkyl and R² is H or alkoxy;
R¹ and R³ are each, independently, C₁-C₆-alkyl groups; or
R¹ is alkyl and R⁴ is alkoxy.

8. (canceled)

9. (canceled)

10. (canceled)

11. (canceled)

12. (canceled)

13. The method of claim 6, wherein;
R⁹ is H;
R⁴ is H: or
R⁵ is H.

14. The method of claim 6, wherein:
X² is N and X³ is CH; or
X² is CH and X³ is CH.

15. (canceled)

16. (canceled)

17. (canceled)

18. (canceled)

19. The method of claim 6, wherein the compound of the Formula (I) is a compound of the Formula (II)-(VII): 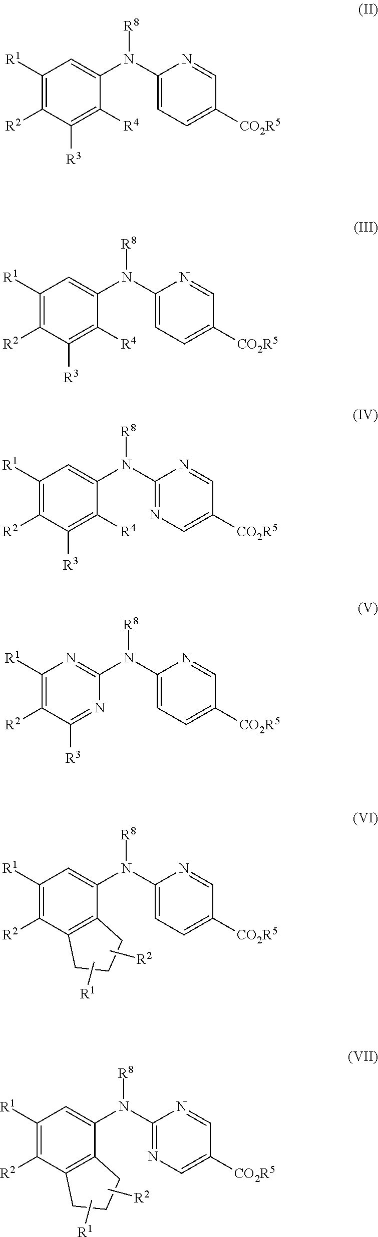
or a pharmaceutically acceptable salt thereof.

20. The method of claim 7, wherein the compound of the Formula (I) is a compound of the formula: 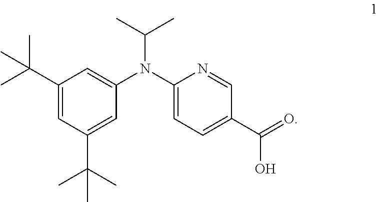 [SMILES: CC(C)N(c1cc(C(C)(C)C)cc(C(C)(C)C)c1)c1ccc(C(=O)O)cn1]
or a pharmaceutically acceptable salt thereof.

21. The method of claim 1, wherein the RAS inhibitor is AMG-510, MRTX 849, captopril, imidapril, zofenopril, candesartan, delapril, telmisartan, aliskiren, moexipril, enalapril, valsartan, fosinopril, irbesartan, perindopril, quinapril, ramipril, eprosartan, olmesartan, trandolapril, losartan, azilsartan, lisinopril, spirapril, benazepril or cilazapril or pharmaceutically acceptable salts thereof; the MEK inhibitor is refametinib, selumetinib, trametinib, cobimetinib, pimasertib, RO-4987655, RO5126766, WX-554, HL-085, PD-0325901, MEK 162, AZD-8330, TAK-733 or GDC-0623 or pharmaceutically acceptable salts thereof; or
the BRAF inhibitor is vemurafenib, dabrafenib, encorafenib or binimetinib or pharmaceutically acceptable salts thereof.

22. (canceled)

23. (canceled)

24. A method for treating plexiform neurofibromas (PNFs) or malignant peripheral nerve sheath tumors (MPNSTs), the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

25. A method of treating RASopathies, the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

26. The method of claim 25, wherein the RASoptathies are neurofibromatosis type 1, Noonan syndrome, Noonan syndrome with multiple lentigines, capillary malformation-arteriovenous malformation syndrome, Costello syndrome, cardio-facio-cutaneous syndrome or Legius syndrome or a combination thereof.

27. A method of treating schwannomas or gliomas, the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

28. The method of claim 27, wherein the glioma is an optical pathway glioma, a low-grade glioma, and/or a high-grade glioma.

29. A method of treating cancer, the method comprising administering a therapeutically effective amount of (i) a single selective RXR agonist; or (ii) the combination of at least one RXR agonist and at least one inhibitor of RAS, MEK, and BRAF or a composition comprising a therapeutically effective amount of (i) a single selective RXR agonist; or (ii) the combination of at least one RXR agonist and at least one inhibitor of RAS, MEK, and BRAF.

30. The method of claim 29, wherein the cancer is rhabdomyosarcoma, neuroblastoma, juvenile myelomonocytic leukemia, gastronintestinal stromal tumors, carcinoid, breast cancer, endocrine cancer, melanoma, acute lymphoblastic leukemia, ovarian cancer, prostate cancer, meningioma, and undifferentiated pleomorphic sarcoma.

31. The method of claim 30, wherein the endocrine cancer is pheochromocytoma or a neuroendocrine tumor.
